SEQUENCE LISTING (99 sequences)

SEQ ID NO: 1 - RNA, 53 nt, Artificial Sequence
  synthetic construct
aaccccuaccaacuggucgggguuugaaacagacaauugugucgcugccaucg

SEQ ID NO: 2 - RNA, 53 nt, Artificial Sequence
  sythetic construct
aaccccuaccaacuggucgggguuugaaacgcggcggccgggaggcuccaugc

SEQ ID NO: 3 - RNA, 56 nt, Artificial Sequence
  synthetic construct
aaccccuaccaacuggucgggguuugaaacagacaauugugucgcugccaucguuu

SEQ ID NO: 4 - RNA, 56 nt, Artificial Sequence
  synthetic construct
aaccccuaccaacuggucgggguuugaaacgcggcggccgggaggcuccaugcuuu

SEQ ID NO: 5 - RNA, 56 nt, Artificial Sequence; identical to SEQ ID NO: 3
  synthetic construct

SEQ ID NO: 6 - RNA, 56 nt, Artificial Sequence; identical to SEQ ID NO: 4
  synthetic construct

SEQ ID NO: 7 - RNA, 56 nt, Artificial Sequence; identical to SEQ ID NO: 3
  synthetic construct

SEQ ID NO: 8 - RNA, 56 nt, Artificial Sequence; identical to SEQ ID NO: 4
  synthetic construct

SEQ ID NO: 9 - DNA, 54 bp, Artificial Sequence
  synthetic construct
aaccccuaccaacuggucgggguuugaaacagacaauugugucgcugccaucgt

SEQ ID NO: 10 - DNA, 54 bp, Artificial Sequence
  synthetic construct
aaccccuaccaacuggucgggguuugaaacgcggcggccgggaggcuccaugct

SEQ ID NO: 11 - RNA, 53 nt, Artificial Sequence; identical to SEQ ID NO: 1
  synthetic construct

SEQ ID NO: 12 - RNA, 53 nt, Artificial Sequence; identical to SEQ ID NO: 2
  synthetic construct

SEQ ID NO: 13 - RNA, 53 nt, Artificial Sequence; identical to SEQ ID NO: 1
  synthetic construct

SEQ ID NO: 14 - RNA, 53 nt, Artificial Sequence; identical to SEQ ID NO: 2
  synthetic construct

SEQ ID NO: 15 - RNA, 53 nt, Artificial Sequence
  synthetic construct
aaccccuaccaacuggucgggguuugaaacuccccgaggaggggcagcgccgc

SEQ ID NO: 16 - RNA, 56 nt, Artificial Sequence
  synthetic construct
aaccccuaccaacuggucgggguuugaaacuccccgaggaggggcagcgccgcuuu

SEQ ID NO: 17 - RNA, 56 nt, Artificial Sequence; identical to SEQ ID NO: 16
  synthetic construct

SEQ ID NO: 18 - RNA, 56 nt, Artificial Sequence; identical to SEQ ID NO: 16
  synthetic construct

SEQ ID NO: 19 - DNA, 54 bp, Artificial Sequence
  synthetic construct
aaccccuaccaacuggucgggguuugaaacuccccgaggaggggcagcgccgct

SEQ ID NO: 20 - RNA, 53 nt, Artificial Sequence; identical to SEQ ID NO: 15
  synthetic construct

SEQ ID NO: 21 - RNA, 53 nt, Artificial Sequence; identical to SEQ ID NO: 15
  synthetic construct

SEQ ID NO: 22 - RNA, 53 nt, Artificial Sequence
  synthetic construct
aaccccuaccaacuggucgggguuugaaaccgagccaggcugaaccggguaua

SEQ ID NO: 23 - RNA, 56 nt, Artificial Sequence
  synthetic construct
aaccccuaccaacuggucgggguuugaaaccgagccaggcugaaccggguauauuu

SEQ ID NO: 24 - RNA, 56 nt, Artificial Sequence; identical to SEQ ID NO: 23
  synthetic construct

SEQ ID NO: 25 - RNA, 56 nt, Artificial Sequence; identical to SEQ ID NO: 23
  synthetic construct

SEQ ID NO: 26 - DNA, 54 bp, Artificial Sequence
  synthetic construct
aaccccuaccaacuggucgggguuugaaaccgagccaggcugaaccggguauat

SEQ ID NO: 27 - RNA, 53 nt, Artificial Sequence; identical to SEQ ID NO: 22
  synthetic construct

SEQ ID NO: 28 - RNA, 53 nt, Artificial Sequence; identical to SEQ ID NO: 22
  synthetic construct

SEQ ID NO: 29 - RNA, 53 nt, Artificial Sequence
  synthetic construct
aaccccuaccaacuggucgggguuugaaacuacuuaccuaaugcgcgguagau

SEQ ID NO: 30 - RNA, 56 nt, Artificial Sequence
  synthetic construct
aaccccuaccaacuggucgggguuugaaacuacuuaccuaaugcgcgguagauuuu

SEQ ID NO: 31 - RNA, 56 nt, Artificial Sequence; identical to SEQ ID NO: 30
  synthetic construct

SEQ ID NO: 32 - RNA, 56 nt, Artificial Sequence; identical to SEQ ID NO: 30
  synthetic construct

SEQ ID NO: 33 - DNA, 54 bp, Artificial Sequence
  synthetic construct
aaccccuaccaacuggucgggguuugaaacuacuuaccuaaugcgcgguagaut

SEQ ID NO: 34 - RNA, 53 nt, Artificial Sequence; identical to SEQ ID NO: 29
  synthetic construct

SEQ ID NO: 35 - RNA, 53 nt, Artificial Sequence; identical to SEQ ID NO: 29
  synthetic construct

SEQ ID NO: 36 - RNA, 53 nt, Artificial Sequence; identical to SEQ ID NO: 2
  synthetic construct

SEQ ID NO: 37 - RNA, 53 nt, Artificial Sequence; identical to SEQ ID NO: 2
  synthetic construct

SEQ ID NO: 38 - RNA, 53 nt, Artificial Sequence; identical to SEQ ID NO: 2
  synthetic construct

SEQ ID NO: 39 - RNA, 56 nt, Artificial Sequence
  synthetic construct
uuuaaccccuaccaacuggucgggguuugaaacgcggcggccgggaggcuccaugc

SEQ ID NO: 40 - RNA, 59 nt, Artificial Sequence
  synthetic construct
uuuaaccccuaccaacuggucgggguuugaaacgcggcggccgggaggcuccaugcuuu

SEQ ID NO: 41 - RNA, 56 nt, Artificial Sequence; identical to SEQ ID NO: 4
  synthetic construct

SEQ ID NO: 42 - RNA, 56 nt, Artificial Sequence; identical to SEQ ID NO: 39
  synthetic construct

SEQ ID NO: 43 - RNA, 53 nt, Artificial Sequence; identical to SEQ ID NO: 1
  synthetic construct

SEQ ID NO: 44 - RNA, 53 nt, Artificial Sequence; identical to SEQ ID NO: 1
  synthetic construct

SEQ ID NO: 45 - RNA, 53 nt, Artificial Sequence; identical to SEQ ID NO: 1
  synthetic construct

SEQ ID NO: 46 - RNA, 56 nt, Artificial Sequence
  synthetic construct
uuuaaccccuaccaacuggucgggguuugaaacagacaauugugucgcugccaucg

SEQ ID NO: 47 - RNA, 59 nt, Artificial Sequence
  synthetic construct
uuuaaccccuaccaacuggucgggguuugaaacagacaauugugucgcugccaucguuu

SEQ ID NO: 48 - RNA, 56 nt, Artificial Sequence; identical to SEQ ID NO: 3
  synthetic construct

SEQ ID NO: 49 - RNA, 56 nt, Artificial Sequence; identical to SEQ ID NO: 46
  synthetic construct

SEQ ID NO: 50 - RNA, 52 nt, Artificial Sequence
  synthetic construct
accccuaccaacuggucgggguuugaaacuacuuaccuaaugcgcgguagau

SEQ ID NO: 51 - RNA, 53 nt, Artificial Sequence; identical to SEQ ID NO: 29
  synthetic construct

SEQ ID NO: 52 - RNA, 53 nt, Artificial Sequence; identical to SEQ ID NO: 29
  synthetic construct

SEQ ID NO: 53 - RNA, 56 nt, Artificial Sequence
  synthetic construct
uuuaaccccuaccaacuggucgggguuugaaacuacuuaccuaaugcgcgguagau

SEQ ID NO: 54 - RNA, 56 nt, Artificial Sequence; identical to SEQ ID NO: 30
  synthetic construct

SEQ ID NO: 55 - RNA, 56 nt, Artificial Sequence; identical to SEQ ID NO: 30
  synthetic construct

SEQ ID NO: 56 - RNA, 56 nt, Artificial Sequence; identical to SEQ ID NO: 53
  synthetic construct

SEQ ID NO: 57 - RNA, 56 nt, Artificial Sequence
  synthetic construct
aaccccuaccaacuggucgggguuugaaacgaucacugaguccuucucgccaguuu

SEQ ID NO: 58 - RNA, 56 nt, Artificial Sequence
  synthetic construct
aaccccuaccaacuggucgggguuugaaaccgaucacagcaauagucccauaguuu

SEQ ID NO: 59 - RNA, 53 nt, Artificial Sequence
  synthetic construct
aaccccuaccaacuggucgggguuugaaacgaucacugaguccuucucgccag

SEQ ID NO: 60 - RNA, 56 nt, Artificial Sequence; identical to SEQ ID NO: 57
  synthetic construct

SEQ ID NO: 61 - RNA, 56 nt, Artificial Sequence; identical to SEQ ID NO: 57
  synthetic construct

SEQ ID NO: 62 - DNA, 54 bp, Artificial Sequence
  synthetic construct
aaccccuaccaacuggucgggguuugaaacgaucacugaguccuucucgccagt

SEQ ID NO: 63 - RNA, 53 nt, Artificial Sequence; identical to SEQ ID NO: 59
  synthetic construct

SEQ ID NO: 64 - RNA, 53 nt, Artificial Sequence; identical to SEQ ID NO: 59
  synthetic construct

SEQ ID NO: 65 - DNA, 53 bp, Artificial Sequence
  synthetic construct
aaccccuaccaacuggucgggguuugaaaccgaucacagcaauagucccauag

SEQ ID NO: 66 - RNA, 56 nt, Artificial Sequence; identical to SEQ ID NO: 58
  synthetic construct

SEQ ID NO: 67 - RNA, 56 nt, Artificial Sequence; identical to SEQ ID NO: 58
  synthetic construct

SEQ ID NO: 68 - DNA, 54 bp, Artificial Sequence
  synthetic construct
aaccccuaccaacuggucgggguuugaaaccgaucacagcaauagucccauagt

SEQ ID NO: 69 - RNA, 53 nt, Artificial Sequence
  synthetic construct
aaccccuaccaacuggucgggguuugaaaccgaucacagcaauagucccauag

SEQ ID NO: 70 - RNA, 53 nt, Artificial Sequence; identical to SEQ ID NO: 69
  synthetic construct

SEQ ID NO: 71 - RNA, 53 nt, Artificial Sequence; identical to SEQ ID NO: 59
  synthetic construct

SEQ ID NO: 72 - RNA, 53 nt, Artificial Sequence; identical to SEQ ID NO: 59
  synthetic construct

SEQ ID NO: 73 - RNA, 53 nt, Artificial Sequence; identical to SEQ ID NO: 59
  synthetic construct

SEQ ID NO: 74 - RNA, 56 nt, Artificial Sequence
  synthetic construct
uuuaaccccuaccaacuggucgggguuugaaacgaucacugaguccuucucgccag

SEQ ID NO: 75 - RNA, 59 nt, Artificial Sequence
  synthetic construct
uuuaaccccuaccaacuggucgggguuugaaacgaucacugaguccuucucgccaguuu

SEQ ID NO: 76 - RNA, 56 nt, Artificial Sequence; identical to SEQ ID NO: 57
  synthetic construct

SEQ ID NO: 77 - RNA, 56 nt, Artificial Sequence; identical to SEQ ID NO: 74
  synthetic construct

SEQ ID NO: 78 - RNA, 53 nt, Artificial Sequence; identical to SEQ ID NO: 69
  synthetic construct

SEQ ID NO: 79 - RNA, 53 nt, Artificial Sequence; identical to SEQ ID NO: 69
  synthetic construct

SEQ ID NO: 80 - RNA, 53 nt, Artificial Sequence; identical to SEQ ID NO: 69
  synthetic construct

SEQ ID NO: 81 - RNA, 56 nt, Artificial Sequence
  synthetic construct
uuuaaccccuaccaacuggucgggguuugaaaccgaucacagcaauagucccauag

SEQ ID NO: 82 - RNA, 59 nt, Artificial Sequence
  synthetic construct
uuuaaccccuaccaacuggucgggguuugaaaccgaucacagcaauagucccauaguuu

SEQ ID NO: 83 - RNA, 56 nt, Artificial Sequence; identical to SEQ ID NO: 58
  synthetic construct

SEQ ID NO: 84 - RNA, 56 nt, Artificial Sequence; identical to SEQ ID NO: 81
  synthetic construct

SEQ ID NO: 85 - DNA, 54 bp, Artificial Sequence
  synthetic construct
aaccccuaccaacuggucgggguuugaaacauaacaaucacagcaaugacccat

SEQ ID NO: 86 - DNA, 53 bp, Artificial Sequence
  synthetic construct
aaccccuaccaacuggucgggguuugaaacauaacaaucacagcaaugaccca

SEQ ID NO: 87 - DNA, 54 bp, Artificial Sequence
  synthetic construct
aaccccuaccaacuggucgggguuugaaacucacaaauaguauggguggcaagt

SEQ ID NO: 88 - RNA, 53 nt, Artificial Sequence
  synthetic construct
aaccccuaccaacuggucgggguuugaaacucacaaauaguauggguggcaag

SEQ ID NO: 89 - DNA, 54 bp, Artificial Sequence
  synthetic construct
aaccccuaccaacuggucgggguuugaaacacuccacuggacagagcugccugt

SEQ ID NO: 90 - RNA, 53 nt, Artificial Sequence
  synthetic construct
aaccccuaccaacuggucgggguuugaaacacuccacuggacagagcugccug

SEQ ID NO: 91 - DNA, 54 bp, Artificial Sequence
  synthetic construct
aaccccuaccaacuggucgggguuugaaacgcaagcccaugguuacuagcguct

SEQ ID NO: 92 - RNA, 53 nt, Artificial Sequence
  synthetic construct
aaccccuaccaacuggucgggguuugaaacgcaagcccaugguuacuagcguc

SEQ ID NO: 93 - DNA, 54 bp, Artificial Sequence
  synthetic construct
aaccccuaccaacuggucgggguuugaaacuccaucauucugaacugccacact

SEQ ID NO: 94 - DNA, 53 bp, Artificial Sequence
  synthetic construct
aaccccuaccaacuggucgggguuugaaacuccaucauucugaacugccacac

SEQ ID NO: 95 - DNA, 54 bp, Artificial Sequence
  synthetic construct
aaccccuaccaacuggucgggguuugaaaccaaguuucaauaggagagcugugt

SEQ ID NO: 96 - RNA, 53 nt, Artificial Sequence
  synthetic construct
aaccccuaccaacuggucgggguuugaaaccaaguuucaauaggagagcugug

SEQ ID NO: 97 - RNA, 53 nt, Artificial Sequence
  synthetic construct
aaccccuaccaacuggucgggguuugaaacagaacagaucuacaagagaucga

SEQ ID NO: 98 - RNA, 53 nt, Artificial Sequence
  synthetic construct
aaccccuaccaacuggucgggguuugaaacaaguugguugguuuguuaccugg

SEQ ID NO: 99 - RNA, 53 nt, Artificial Sequence
  synthetic construct
aaccccuaccaacuggucgggguuugaaacacaagagaucgaaaguugguugg

(19) United States
(12) Patent Application Publication
(10) Pub. No.: US 2024/0043840 A1
(43) Pub. Date: Feb. 8, 2024

(54) CHEMICALLY MODIFIED CRISPR-CAS13 GUIDE RNAS

(71) Applicants: New York Genome Center, Inc., New York, NY (US); New York University, New York, NY (US)

(72) Inventors: Neville E. Sanjana, New York, NY (US); Hans-Hermann Wessels, Brooklyn, NY (US); Alejandro Mendez-Mancilla, Brooklyn, NY (US); Mateusz Legut, Jersey City, NJ (US)

(21) Appl. No.: 18/257,504

(22) PCT Filed: Dec. 14, 2021
(86) PCT No.: PCT/US2021/063314

§ 371 Date: Jun. 14, 2023

Related U.S. Application Data
(60) Provisional application No. 63/148,966, filed on Feb. 12, 2021.
(60) Provisional application No. 63/125,220, filed on Dec. 14, 2020.

(51) Int. Cl. C12N 15/113; C12N 15/11

(52) U.S. Cl. CPC C12N 15/1131; C12N 15/11; C12N 15/111; C12N 2310/20; C12N 2310/315; C12N 2310/346; C12N 2310/317; C12N 2310/344

(57) ABSTRACT

Provided herein are compositions and methods comprising modified crRNA comprising a spacer sequence and a direct repeat sequence, wherein the crRNA comprises one or more chemically modified nucleotides. Also provided are methods of enhancing modulation of gene transcripts in a cell, the method comprising introducing into the cell a modified crRNA as described herein, and a Cas13 polypeptide, an mRNA encoding a Cas13 polypeptide, and/or a recombinant expression vector comprising a nucleotide sequence encoding a Cas13 polypeptide, wherein the modified crRNA guides the Cas13 polypeptide to the target RNA sequence, and wherein the modified crRNA induces regulation of the target RNA with an enhanced activity relative to a corresponding unmodified crRNA.

### STATEMENT OF GOVERNMENT SUPPORT

This invention was made with government support under HGO10099 and CA218668 awarded by the National Institutes of Health. The government has certain rights in the invention.

### BACKGROUND

The CRISPR Cas13 enzyme family has shown remarkable versatility in basic biology and biotechnological applications¹, including efficient RNA knockdown¹⁻⁴, RNA-editing and other transcript modifications^{4,5}, live imaging^{6,7} and diagnostics⁸⁻¹⁰. Importantly, CRISPR-based transcriptome modulation has been proposed to offer considerable therapeutic potential for a wide spectrum of RNA-mediated diseases^{1,11,12}. In all cases, the Cas13-mediated effects are directed by CRISPR RNAs (crRNA) that guide Cas13 to the target RNA by RNA-RNA hybridization of a short spacer sequence (˜20-30 nt) to the target site. Cas13-based effectors act on the RNA level and do not result in permanent changes to the genome. However, without a continuous source of crRNA expression, effects are very short-lived due to rapid crRNA degradation by endogenous RNA nucleases and regeneration of the cellular steady-state by continuous target RNA expression. Although continuous crRNA expression can be achieved via genetic manipulation, such as viral vectors expressing the crRNA and/or Cas13^{2,3,13} these methods are less desirable for more physiologically-relevant or therapeutic settings. Therefore, one of the main challenges for transcriptome manipulations is to achieve efficient and precise delivery of CRISPR systems for robust RNA manipulation without modifying the host DNA sequence. Although, recent work has shown that in vitro transcribed crRNAs and recombinant Cas13 proteins can be injected into zebrafish embryos¹⁴, it remains unclear if a similar approach in human cells can lead to lasting effects.

What is needed are crRNAs that extend transient Cas13-mediated effects.

### SUMMARY OF THE INVENTION

Provided herein in a first aspect, is a modified crRNA comprising a spacer sequence and a direct repeat sequence. The crRNA comprises one or more chemically modified nucleotides. In one embodiment, the spacer sequence comprises at least 23 nucleotides, and is complementary to a target RNA sequence. In one embodiment, the crRNA comprises 1, 2, 3, 4, or 5 modified nucleotides at the 3′ end of the spacer sequence. In one embodiment, the chemically modified nucleotide comprises a modification selected from 2′-O-methyl (M), 3′phosphorothioate (S), 2′O-methyl-3′-phosphorothioate (MS) and 3′ Inverted thymine (invT).

In another aspect, a method of enhancing the stability of a crRNA is provided. The method includes modifying a crRNA comprising a spacer sequence and a direct repeat sequence, with one or more chemically modified nucleotides. In one embodiment, the one or more chemically modified nucleotides comprises a chemical modification selected from 2′-O-methyl (M), 3′phosphorothioate (S), 2′O-methyl-3′-phosphorothioate (MS) and 3′ inverted thymine (invT).

In yet another aspect, a method of enhancing modulation of gene transcripts in a cell is provided. The method includes introducing into the cell a modified crRNA as described herein, and a Cas13 polypeptide, an mRNA encoding a Cas13 polypeptide, and/or a recombinant expression vector comprising a nucleotide sequence encoding a Cas13 polypeptide, wherein the modified crRNA guides the Cas13 polypeptide to the target RNA sequence, and wherein the modified crRNA induces regulation of the target RNA with an enhanced activity relative to a corresponding unmodified crRNA. In one embodiment, the enhanced activity comprises increased stability of the modified crRNA and/or increased specificity of the modified crRNA for the target RNA.

In another aspect, a method of treating COVID-19 infection is provided. The method includes introducing into a subject having, or suspected of having, COVID-19 infection a crRNA composition as described herein, and a Cas13 polypeptide, an mRNA encoding a Cas13 polypeptide, and/or a recombinant expression vector comprising a nucleotide sequence encoding a Cas13 polypeptide, wherein the modified crRNA guides the Cas13 polypeptide to the SARS-CoV-2 leader sequence, and wherein the modified crRNA induces knockdown of the SARS-CoV-2 virus, thereby treating COVID-19 infection.

Other aspects and advantages of the invention will be readily apparent from the following detailed description of the invention.

### BRIEF DESCRIPTION OF THE DRAWINGS

FIGS. 1A-1E demonstrate that chemically modified crRNAs improve Cas13 knockdown efficacy in human cells. (a) Chemical modifications incorporated during chemical synthesis of crRNAs and schematic structures of the chemically modified crRNAs, nucleotides with chemical modifications are marked and placed at the 3′ crRNA (sequences can be found in FIG. 7A-7D). (b) Representative flow cytometry results for CD46 knockdown in HEK293FT-TetO-RfxCas13d-NLS cells nucleofected with respective synthetic crRNAs targeting CD46. (c, d, e) Percent of CD46, CD55 and CD71 protein expression 72 hours post-nucleofection with the indicated crRNAs in HEK293FT-TetO-RfxCas13d-NLS cells. Protein surface expression was measured by flow cytometry, c) synthetic crRNAs with chemical modification located in the 3′ crRNA end or d) synthetic crRNAs with 3′-O-methyl (M) modification in the nucleotides along the crRNA sequence or in the guide sequence only e) synthetic crRNAs chemically modified with a phosphorothioate bond (S) placed in the crRNA sequence at the 5′, or 3′ end or at both ends or in three uridines added at the 5′, 3′ and both (Se). Relative protein expression was calculated comparing the normalized median fluorescent intensity percentage (MFI %) to cells nucleofected with non-targeting (NT) crRNA. Bars represent mean values ±sd, n=3.

FIGS. 2A-2C show transcript knockdown with chemically-modified crRNAs is sustained over multiple days. (a) Schematics of experimental design to test the temporal dynamics of transient CD46 knockdown conferred by nucleofecting with indicated crRNAs in the HEK293FT-TetO-RfxCas13d-NLS cells. b) Representative of CD46 histograms upon target knockdown after three days (top), two days (middle) and one day (bottom). c) Percent CD46 expression relative to cells nucleofected with non-targeting (NT) crRNAs (n=3 biological replicates). Error bars, s.d.

FIG. 2D shows a schematic of experimental approach to measure SARS-CoV-2 RNA knockdown using a single reporter TRSLeader-SARS-CoV-2-GFPd2PEST. FIG. 2E shows GFP expression in HEK293FT-TetO-RfxCad13d-NLS cells. “GFP”: pool of three crRNAs targeting eGFP; “g1”, “g2” and “g3”: crRNAs targeting the SARS-CoV-2 TRS-Leader sequence; “All”: g1, g2 and g3 pool. Percent GFP expression is determined relative to cells nucleofected with 0.4 μg of TRSLeader-SARS-CoV-2-d2eGFP plasmid and 7.5 μM of synthetic crRNA, cells were plated 24 hours before nucleofection with 1 μg/mL of doxycycline, median fluorescent intensity percentage (MFI %) was calculated taking as reference the non-targeting (NT) crRNA conditions. Bars represent media values ±sd, n=3. FIG. 2E shows cells transfected with leader sequence targeting gRNAs showed significant reduction of viral N protein 24 hours post infection.

FIGS. 3A-3E demonstrate that Cas13d ribonucleoprotein (RNP) complexes with chemically-modified crRNA lead to robust knockdown in human cell lines and primary immune cells. (a) Denaturing RNA gel showing the cleavage activity of the indicated RfxCas13d proteins tagged with either one (sTag) or two (dTag) affinity purification tags and their cleavage activity using chemically modified crRNA (3'S) targeting CD46. RfxCas13d-sTag includes only a C-terminal HA tag. RfxCas13d-dTag includes C-terminal HA and 6xHis tags and an N-terminal MKIEE solubility sequence. (b) CD46 expression in HEK293FT cells at 24 and 48 hours after nucleofection with Cas13 RNP complexed at the indicated temperature prior to nucleofection. (c) CD46 expression in HEK293FT cells at 24 hours after nucleofection with synthetic crRNAs only and Cas13 RNP complexes with different protein amounts (1, 5, 10 and 20 μg) complexed with CD46-targeting synthetic crRNAs with no chemical modification or with a phosphorothioate bond at the 3′ end (3'S). (d) Leukapheresis and activation of primary human T cells prior to Cas13 RNP electroporation. (e) CD46 expression in primary human T cells (CD4+ and CD8+) at 24 hours after nucleofection with Cas13 RNP complexed with CD46-targeting synthetic crRNAs with no chemical modification or with a 3'S modification. Bars represent mean values ±sd, n =3.

FIGS. 4A and 4B demonstrate optimization for Cas13 synthetic crRNAs knockdown in mammalian cells. (a) Flow cytometry gating strategy for CD46, CD55 and CD71 knockdown in HEK293FT-Tet-NLS-RfxCas13d-NLS cells nucleofected with synthetic crRNAs. (b) Representative histograms of CD55 and CD71 expression in HEK293FT-Tet-RfxCas13d-NLS cells nucleofected with the indicated synthetic crRNAs.

FIGS. 5A to 5C show a comparison of Cas13 CRISPR RNA (crRNA) knockdown efficiencies with guide sequence prediction and timing of Cas13 induction. (a) Predicted Cas13 guide RNA (gRNA) scores targeting CD46, CD55 and CD71. For each target, we used the cas13design webtool1 to design 2 guide RNAs one predicted as low activity (Q1) and one predicted as high activity (Q4). (b) Expression of CD46, CD55 and CD71 in HEK293FTTet-RfxCas13d-NLS cells nucleofected with the indicated synthetic crRNAs (n=3 nucleofection replicates). Bars indicate the mean and error bars denote s.d. (c) CD46 knockdown in HEK293FTTet-NLS-RfxCas13d-NLS cells with different media and timing of doxycycline induction (n=2 nucleofection replicates). Cells were grown in standard D10 media (top) or Tet-free D10 media (bottom) and doxycycline was added either 1 day prior to and/or upon crRNA nucleofection. Bars indicate the mean and error bars denote s.d.

FIGS. 6A-6E show characterization of Cas13 ribonucleoproteins (RNPs) in vitro and in vivo. (a) Denaturing RNA gel showing the cleavage activity of recombinant RfxCas13d proteins tagged with C-terminal HA tag (single-tag, or sTag), or with N-terminal MKIEE and C-terminal HA and 6xHis tags (double-tag, or dTag) and their cleavage activity using a chemically-modified crRNA (3'S) targeting CD46. Two independent purifications are shown for each enzyme. (b) Denaturing RNA gel showing recombinant RfxCas13d proteins tagged with C-terminal HA tag (single-tag, or sTag) or with C-terminal HA tag (single-tag, or sTag), or with N-terminal MKIEE, C-terminal HA and 6xHis tags (double-tag, or dTag) and their cleavage activity using a chemically-modified crRNA (3'S) targeting CD46. Two independent purifications are shown for each enzyme. (c) Denaturing RNA gel showing recombinant RfxCas13d proteins tagged with C-terminal HA tag (single-tag, or sTag) or with N-terminal MKIEE, C-terminal HA and 6xHis tags (double-tag, or dTag) and their cleavage activity using a chemically-modified crRNA (3'S) targeting CD46. Two independent purifications are shown for each enzyme. (d) Flow cytometry gating strategy for CD46 knockdown using RfxCas13d-dTag RNPs with synthetic crRNAs in HEK293FT cells. (e) Flow cytometry gating strategy for CD46 knockdown using RfxCas13d-sTag RNPs with synthetic crRNAs in human primary CD4+ and CD8+ T cells.

FIGS. 7A-7D show the sequences of the crRNAs used herein.

### DETAILED DESCRIPTION OF THE INVENTION

Here, it is shown that chemically synthesized crRNAs can be used for RNA editing, and it is demonstrated that utilizing chemically modified crRNAs significantly enhances Cas13 RNA targeting in human cell lines and primary T cells. Furthermore, the applicability of Cas13 RNPs complexes together with chemically modified crRNAs is demonstrated in human cells and primary cells. In conclusion, it is demonstrated that chemically modified crRNAs represent a relevant alternative to improve CRISPR-Cas13 RNA editing properties in a large set of biotechnological and therapeutic applications.

RNA-targeting CRISPR-Cas13 proteins have recently emerged as a powerful platform to transiently modulate gene expression outcomes. However, protein and CRISPR-RNA (crRNA) delivery in human cells can be challenging and knockdown can be transient due to rapid crRNA degradation. Here, it is shown that synthetic crRNAs improve RNA targeting efficiency and half-life in human cells. It is also demonstrated herein that co-delivery of modified crRNAs and recombinant Cas13 enzyme in ribonucleoprotein (RNP) complexes enables transient gene expression modulation in primary CD4+ and CD8+ T-cells. This system represents a robust and efficient method to transiently modulate transcripts without genetic manipulation.

Provided herein are compositions and methods incorporating modified guide RNAs for use with CRISPR Cas13 RNA-targeting systems. Unlike Cas9, Cas13 binds and cleaves RNA rather than DNA substrates. Cas13, uses a short ˜23 base crRNA that interacts with the Cas13 molecule through a uracil-rich stem loop and facilitates target binding and cleavage through a series of conformational changes in the Cas13 molecule. The crRNA is sometimes referred to herein as a guide RNA or gRNA. As noted above, crRNA delivery in human cells can be challenging and the transient effects can be limited in half-life. Thus, provided herein, in a first aspect, are modified crRNAs which provide increased targeting efficiency and/or half-life in human cells. In other cases, the modified crRNAs provide improved activity and/or specificity compared to their unmodified sequence equivalents.

Also provided herein are methods for inducing regulation of a target RNA in a cell. The invention includes using modified Crispr RNAs (crRNAs) that enhance transcript modulation of a target nucleic acid in a primary cell (e.g., cultured in vitro for use in ex vivo therapy) or in a cell in a subject such as a human (e.g., for use in in vivo therapy). The present invention also provides methods for preventing or treating a disease in a subject by targeting a gene transcript or RNA sequence associated with the disease. The present invention can be used with any cell type and at any gene locus that is amenable to modulation of gene expression.

The crRNA provided herein comprises approximately 64 nucleotides, which includes a “spacer” sequence of about 23 nucleotides, that is complementary to a portion of the target sequence. As used herein, a “target sequence” refers to a sequence to which a guide (spacer) sequence is designed to have complementarity, where hybridization between a target sequence and a spacer sequence promotes the formation of a CRISPR complex. A target sequence may comprise RNA polynucleotides. The term “target RNA” refers to an RNA polynucleotide being or comprising the target sequence. In other words, the target RNA may be a RNA polynucleotide or a part of a RNA polynucleotide to which a part of the crRNA, i.e. the spacer sequence, is designed to have complementarity and to which the effector function mediated by the complex comprising CRISPR effector protein and a gRNA is to be directed. In some embodiments, a target sequence is located in the nucleus or cytoplasm of a cell.

In one embodiment, the cell is a primary cell. The term “primary cell” refers to a cell isolated directly from a multicellular organism. Primary cells typically have undergone very few population doublings and are therefore more representative of the main functional component of the tissue from which they are derived in comparison to continuous (tumor or artificially immortalized) cell lines. In some cases, primary cells are cells that have been isolated and then used immediately. In other cases, primary cells cannot divide indefinitely and thus cannot be cultured for long periods of time in vitro. In other embodiments, the cell is a cultured cell, or cell from an organism other than a human or the animal being treated, such as, e.g., a viral cell or bacterial cell. In one embodiment, the cell is a SARS-CoV-2 cell.

In some embodiments, the primary cell is isolated from an organism prior to introducing the modified crRNA and the Cas polypeptide into the primary cell. In certain instances, the primary cell is selected from the group consisting of a stem cell, an immune cell, and a combination thereof. Non-limiting examples of stem cells include hematopoietic stem and progenitor cells (HSPCs) such as CD34+ HSPCs, mesenchymal stem cells, neural stem cells, organ stem cells, and combinations thereof. Non-limiting examples of immune cells include T cells (e.g., CD3+ T cells, CD4+ T cells, CD8+ T cells, tumor infiltrating cells (TILs), memory T cells, memory stem T cells, effector T cells), natural killer cells, monocytes, peripheral blood mononuclear cells (PBMCs), peripheral blood lymphocytes (PBLs), and combinations thereof. In other embodiments, the primary cell or a progeny thereof (e.g., a cell derived from the primary cell) is returned (e.g., administered via any acceptable delivery system and delivery route) to the multicellular organism (e.g., human) after introducing the modified crRNA and the Cas polypeptide into the primary cell.

The nucleic acid sequence of the modified crRNA can be any polynucleotide sequence having sufficient complementarity with a target polynucleotide sequence (e.g., target RNA sequence) to hybridize with the target sequence and direct sequence-specific binding of a CRISPR complex to the target sequence. In some embodiments, the degree of complementarity between a spacer sequence of the modified crRNA and its corresponding target sequence, when optimally aligned using a suitable alignment algorithm, is about or more than about 50%, 60%, 75%, 80%, 85%, 90%, 95%, 97.5%, 99%, or more. The term “complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence. As used herein the term “complementary” may refer to sequences having perfect complementarity, i.e., all the contiguous residues of a nucleic acid sequence will hydrogen bond with the same number of contiguous residues in a second nucleic acid sequence, or substantially complementary sequences, i.e., those having less than 100% complementarity, provided that the two nucleic acids hybridize under stringent conditions. The term “stringent conditions” for hybridization refers to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part 1, Second Chapter “Overview of principles of hybridization and the strategy of nucleic acid probe assay”, Elsevier, N.Y. Optimal alignment may be determined with the use of any suitable algorithm for aligning sequences, non-limiting example of which include ClustalW and Clustal X. In some embodiments, a spacer sequence is about 20, 21, 22, 23, 24, 25, 26, 27, 28, 29, 30, 31, 32, 33, 34, 35, 40, 45, 50, 75, or more nucleotides in length. In some instances, a spacer sequence is about 20 nucleotides in length. In other instances, a spacer sequence is 23 nucleotides in length. In other instances, a spacer sequence is about 25 nucleotides in length. The ability of a spacer sequence to direct sequence-specific binding of a CRISPR complex to a target sequence may be assessed by any suitable assay. For example, the components of a CRISPR system sufficient to form a CRISPR complex, including the spacer sequence to be tested, may be provided to a host cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of a CRISPR complex, including the spacer sequence to be tested and a control spacer sequence different from the test spacer sequence, and comparing binding or rate of cleavage at the target sequence between the test and control spacer sequence reactions. The nucleotide sequence of a modified crRNA can be selected using any of the web-based software known in the art, including the Cas13 guide designer provided by the Sanjana lab, available at http://cas13design.nygenome.org.

The crRNA also includes a short hairpin region, also referred to as a “direct repeat” (DR) sequence, via which the Cas13 protein complexes with the guide RNA. Like all CRISPR-Cas effectors described to date, Cas13 enzymes each recognize a direct repeat (DR) sequence containing a conserved stem loop structure within their cognate crRNA. However, between Cas13 subtypes, the DR sequence motifs, RNA fold, and DR position relative to the spacer sequence are each distinct. For Cas13a and Cas13d, the DR is located on the 5′ end while the Cas13b DR is 3′ of the spacer sequence. Thus, in one embodiment, the DR is 5′ to the spacer sequence. In another embodiment, the DR is 3′ to the spacer sequence. See, e.g., Cheng et al, Structural Basis for the RNA-Guided Ribonuclease Activity of CRISPR-Cas13d, Cell. 2018 Sep. 20; 175(1):212-223.e17, which is incorporated herein by reference.

The modified crRNAs provided herein include a chemical modification in one or more nucleotides. For instance, a spacer sequence that is about 23 nucleotides in length may have 1 or more, e.g., 1, 2, 3, 4, 5, 6, 7, 8, 9, or 10 more modified nucleotides, or a direct repeat sequence that is about 30 nucleotides in length may have 1 or more, e.g., 1, 2, 3, 4, 5, 6, 7, 8, 9, or 10 more modified nucleotides. The term “modified nucleotide” refers to a nucleotide that contains one or more chemical modifications (e.g., substitutions), in or on the nitrogenous base of the nucleoside (e.g., cytosine (C), thymine (T) or uracil (U), adenine (A) or guanine (G)), in or on the sugar moiety of the nucleoside (e.g., ribose, deoxyribose, modified ribose, modified deoxyribose, six-membered sugar analog, or open-chain sugar analog), or the phosphate. In some cases, the spacer sequence includes at least 2, 3, 4, 5, 6, 7, 8, 9, 10, or more modified nucleotides. The modified nucleotides can be located at any nucleic acid position of the crRNA sequence. The chemical modification can be in the direct repeat, the spacer sequence, or both. Further, in other embodiments, the crRNA includes an “extension” of one or more modified nucleotides at the 5′ or 3′ end. In one embodiment, the modified nucleotides can be at or near the first (5′) and/or last (3′) nucleotide of the spacer sequence, and/or at any position in between. In a particular embodiment, the modified nucleotides are at the 3′ end of the spacer sequence. For example, for a guide sequence that is 23 nucleotides in length, the one or more modified nucleotides can be located at nucleic acid position 1, position 2, position 3, position 4, position 5, position 6, position 7, position 8, position 9, position 10, position 11, position 12, position 13, position 14, position 15, position 16, position 17, position 18, position 19, position 20, position 21, position 22, and/or position 23 of the spacer sequence (read from 5′ to 3′). In one embodiment, the modified nucleotides are located in position 21, 22, and/or 23 of the spacer sequence. In another embodiment, the modified nucleotides are located at the last 1, 2 or 3 terminal nucleotides at the 3′ end.

In another embodiment, the modified nucleotides can be at or near the first (5′) and/or last (3′) nucleotide of the direct repeat sequence, and/or at any position in between. In a particular embodiment, the modified nucleotides are at the 3′ end of the direct repeat sequence. For example, for a direct repeat sequence that is 30 nucleotides in length, the one or more modified nucleotides can be located at nucleic acid position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, and/or position 30 of the direct repeat sequence (read from 5′ to 3′). In one embodiment, the modified nucleotides are located in position 1, 2, and/or 3 of the spacer sequence. In another embodiment, the modified nucleotides are located at the first 1, 2 or 3 terminal nucleotides at the 5′ end.

In another embodiment, one or more modified nucleotides are added to the 3′ end of the spacer sequence. In one embodiment, 1, 2, 3, 4, 5, 6 or more modified nucleotides are added to the 3′ end of the spacer sequence. In another embodiment, one or more modified nucleotides are added to the 5′ end of the direct repeat sequence. In one embodiment, 1, 2, 3, 4, 5, 6 or more modified nucleotides are added to the 5′ end of the direct repeat sequence.

The modified nucleotides of the crRNA can include a modification in the ribose (e.g., sugar) group, phosphate group, nucleobase, or any combination thereof. Such modifications include those known in the art, and those described herein. In some embodiments, the modification in the ribose group comprises a modification at the 2′ position of the ribose.

In some embodiments, the phosphate backbone of the modified crRNA is altered. The modified crRNA can include one or more phosphorothioate (S), phosphoramidate (e.g., N3′-P5′-phosphoramidate (NP)), 2′-O-methoxy-ethyl (2′MOE), 2′-O-methyl-ethyl (2′ME), and/or methylphosphonate linkages. In some embodiments, the one or more modified nucleotides in the modified crRNA comprise phosphorothioate (S) linkages.

In some instances, the modification at the 2′ position of the ribose group is selected from 2′-O-methyl (M), 2′-fluoro, 2′-deoxy, and 2′-O-(2-methoxyethyl). In particular embodiments, the one or more modified nucleotides in the modified crRNA comprise 2′-O-methyl (M) nucleotides or 2′-O-methyl 3′-phosphorothioate (MS) nucleotides. In some embodiments, the one or more modified nucleotides in the modified crRNA comprise an inverted thymine (invT) at the 3′ terminus. In one embodiment, combinations of the modifications set forth herein are utilized.

In some embodiments, the modified crRNA includes one or more M nucleotides. In some embodiments, the modified crRNA includes one or more S nucleotides. In some embodiments, the modified crRNA includes one or more MS nucleotides. In some embodiments, the modified crRNA includes an inverted thymine at the 3′ end. In one embodiment, the crRNA includes 1 M nucleotide at the 3′ end of the crRNA. In one embodiment, the crRNA includes 2 M nucleotides at the 3′ end of the crRNA. In another embodiment, the crRNA includes 3 M nucleotides at the 3′ end of the crRNA. In one embodiment, the crRNA includes 1 MS nucleotide at the 3′ end of the crRNA. In one embodiment, the crRNA includes 2 MS nucleotides at the 3′ end of the crRNA. In another embodiment, the crRNA includes 3 MS nucleotides at the 3′ end of the crRNA. In one embodiment, the crRNA includes 1 S nucleotide at the 3′ end of the crRNA. In one embodiment, the crRNA includes 2 S nucleotides at the 3′ end of the crRNA. In another embodiment, the crRNA includes 3 S nucleotides at the 3′ end of the crRNA. In one embodiment, the crRNA includes 1 M nucleotide at the 5′ end of the crRNA. In one embodiment, the crRNA includes 2 M nucleotides at the 5′ end of the crRNA. In another embodiment, the crRNA includes 3 M nucleotides at the 5′ end of the crRNA. In one embodiment, the crRNA includes 1 MS nucleotide at the 5′ end of the crRNA. In one embodiment, the crRNA includes 2 MS nucleotides at the 5′ end of the crRNA. In another embodiment, the crRNA includes 3 MS nucleotides at the 5′ end of the crRNA. In one embodiment, the crRNA includes 1 S nucleotide at the 5′ end of the crRNA. In one embodiment, the crRNA includes 2 S nucleotides at the 5′ end of the crRNA. In another embodiment, the crRNA includes 3 S nucleotides at the 5′ end of the crRNA. In each of the embodiments described herein, the nucleotides may be included as part of the crRNA, or as an extension 5′ or 3′ to the crRNA. It is specifically contemplated that combinations of 5′ and 3′ modifications can be used in conjunction in the same crRNA.

Modified nucleotides or nucleotide analogues can include sugar- and/or backbone-modified ribonucleotides (i.e., include modifications to the phosphate-sugar backbone). For example, the phosphodiester linkages of a native or natural RNA may be modified to include at least one of a nitrogen or sulfur heteroatom. In some backbone-modified ribonucleotides the phosphoester group connecting to adjacent ribonucleotides may be replaced by a modified group, e.g., of phosphothioate group. In some sugar-modified ribonucleotides, the 2′ moiety is a group selected from H, OR, R, halo, SH, SR, NH2, NHR, NR2 or ON, wherein R is C1-C6 alkyl, alkenyl or alkynyl and halo is F, Cl, Br or I.

In some embodiments, the modified nucleotide contains a sugar modification. Non-limiting examples of sugar modifications include 2′-deoxy-2′-fluoro-oligoribonucleotide (2′-fluoro-2′-deoxycytidine-5′-triphosphate, 2′-fluoro-2′-deoxyuridine-5′-triphosphate), 2′-deoxy-2′-deamine oligoribonucleotide (2′-amino-2′-deoxycytidine-5′-triphosphate, 2′-amino-2′-deoxyuridine-5′-triphosphate), 2′-O-alkyl oligoribonucleotide, 2′-deoxy-2′-C-alkyl oligoribonucleotide (2′-0-methylcytidine-5′-triphosphate, 2′-methyluridine-5′-triphosphate), 2′-C-alkyl oligoribonucleotide, and isomers thereof (2′-aracytidine-5′-triphosphate, 2′-arauridine-5′-triphosphate), azidotriphosphate (2′-azido-2′-deoxycytidine-5′-triphosphate, 2′-azido-2′-deoxyuridine-5′-triphosphate), and combinations thereof.

In some embodiments, the modified crRNA contains one or more 2′-fluro, 2′-amino and/or 2′-thio modifications. In some instances, the modification is a 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine, 5-amino-allyl-uridine, 5-bromo-uridine, 5-iodo-uridine, 5-methyl-cytidine, ribo-thymidine, 2-aminopurine, 2′-amino-butyryl-pyrene-uridine, 5-fluoro-cytidine, and/or 5-fluoro-uridine.

There are more than 96 naturally occurring nucleoside modifications found on mammalian RNA. See, e.g., Limbach et al., Nucleic Acids Research, 22(12):2183-2196 (1994). The preparation of nucleotides and modified nucleotides and nucleosides are well-known in the art and described in, e.g., U.S. Pat. Nos. 4,373,071, 4,458,066, 4,500,707, 4,668,777, 4,973,679, 5,047,524, 5,132,418, 5,153,319, 5,262,530, and 5,700,642. Numerous modified nucleosides and modified nucleotides that are suitable for use as described herein are commercially available.

The nucleoside can be an analogue of a naturally occurring nucleoside. In some cases, the analogue is dihydrouridine, methyladenosine, methylcytidine, methyluridine, methylpseudouridine, thiouridine, deoxycytodine, and deoxyuridine.

In some cases, the modified crRNA described herein includes a nucleobase-modified ribonucleotide, i.e., a ribonucleotide containing at least one non-naturally occurring nucleobase instead of a naturally occurring nucleobase. Non-limiting examples of modified nucleobases which can be incorporated into modified nucleosides and modified nucleotides include m5C (5-methylcytidine), m5U (5-methyluridine), m6A (N6-methyladenosine), s2U (2-thiouridine), Um (2′-O-methyluridine), m1A (1-methyl adenosine), m2A (2-methyladenosine), Am (2-1-O-methyladenosine), ms2m6A (2-methylthio-N6-methyladenosine), i6A (N6-isopentenyl adenosine), ms2i6A (2-methylthio-N6isopentenyladenosine), io6A (N6-(cis-hydroxyisopentenyl) adenosine), ms2io6A (2-methylthio-N6-(cis-hydroxyisopentenyl)adenosine), g6A (N6-glycinylcarbamoyladenosine), t6A (N6-threonyl carbamoyladenosine), ms2t6A (2-methylthio-N6-threonyl carbamoyladenosine), m6t6A (N6-methyl-N6-threonylcarbamoyladenosine), hn6A(N6-hydroxynorvalylcarbamoyl adenosine), ms2hn6A (2-methylthio-N6-hydroxynorvalyl carbamoyladenosine), Ar(p) (2-O-ribosyladenosine(phosphate)), I (inosine), m11 (1-methylinosine), m′Im (1,2′-O-dimethylinosine), m3C (3-methylcytidine), Cm (2T-O-methylcytidine), s2C (2-thiocytidine), ac4C (N4-acetylcytidine), f5C (5-fonnylcytidine), m5Cm (5,2-O-dimethylcytidine), ac4Cm (N4acetyl2TOmethylcytidine), k2C (lysidine), m1G (1-methylguanosine), m2G (N2-methylguanosine), m7G (7-methylguanosine), Gm (2′-O-methylguanosine), m22G (N2,N2-dimethylguanosine), m2Gm (N2,2′-O-dimethylguanosine), m22Gm (N2,N2,2′-O-trimethylguanosine), Gr(p) (2′-O-ribosylguanosine(phosphate)), yW (wybutosine), o2yW (peroxywybutosine), OHyW (hydroxywybutosine), OHyW* (undermodified hydroxywybutosine), imG (wyosine), mimG (methylguanosine), Q (queuosine), oQ (epoxyqueuosine), galQ (galtactosyl-queuosine), manQ (mannosyl-queuosine), preQo (7-cyano-7-deazaguanosine), preQi (7-aminomethyl-7-deazaguanosine), G (archaeosine), D (dihydrouridine), m5Um (5,2′-O-dimethyluridine), s4U (4-thiouridine), m5s2U (5-methyl-2-thiouridine), s2Um (2-thio-2′-O-methyluridine), acp3U (3-(3-amino-3-carboxypropyl)uridine), ho5U (5-hydroxyuridine), mo5U (5-methoxyuridine), cmo5U (uridine 5-oxyacetic acid), mcmo5U (uridine 5-oxyacetic acid methyl ester), chm5U (5-(carboxyhydroxymethyl)uridine)), mchm5U (5-(carboxyhydroxymethyl)uridine methyl ester), mcm5U (5-methoxycarbonyl methyluridine), mcm5Um (S-methoxycarbonylmethyl-2-O-methyluridine), mcm5s2U (5-methoxycarbonylmethyl-2-thiouridine), nm5 s2U (5-aminomethyl-2-thiouridine), mnm5U (5-methylaminomethyluridine), mnm5s2U (5-methylaminomethyl-2-thiouridine), mnm5se2U (5-methylaminomethyl-2-selenouridine), ncm5U (5-carbamoylmethyl uridine), ncm5Um (5-carbamoylmethyl-2′-O-methyluridine), cmnm5U (5-carboxymethylaminomethyluridine), cnmm5Um (5-carboxymethylaminomethyl-2-L-Omethyluridine), cmnm5s2U (5-carboxymethylaminomethyl-2-thiouridine), m62A (N6,N6-dimethyladenosine), Tm (2′-O-methylinosine), m4C (N4-methylcytidine), m4Cm (N4,2-O-dimethylcytidine), hm5C (5-hydroxymethylcytidine), m3U (3-methyluridine), cm5U (5-carboxymethyluridine), m6Am (N6,T-O-dimethyladenosine), m62Am (N6,N6,0-2-trimethyladenosine), m2′7G (N2,7-dimethylguanosine), m2′2′7G (N2,N2,7-trimethylguanosine), m3Um (3,2T-O-dimethyluridine), m5D (5-methyldihydrouridine), f5Cm (5-formyl-2′-O-methylcytidine), m1Gm (1,2′-O-dimethylguanosine), m′Am (1,2-O-dimethyl adenosine)irinomethyluridine), tm5s2U (S-taurinomethyl-2-thiouridine)), imG-14 (4-demethyl guanosine), imG2 (isoguanosine), or ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C1-C6)-alkyluracil, 5-methyluracil, 5-(C2-C6)-alkenyluracil, 5-(C2-C6)-alkynyluracil, 5-(hydroxymethyl)uracil, 5-chlorouracil, 5-fluorouracil, 5-bromouracil, 5-hydroxy cytosine, 5-(C1-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, and combinations thereof.

The modified crRNA can be synthesized by any method known to one of ordinary skill in the art. In some embodiments, the modified crRNA is chemically synthesized. Modified crRNAs can be synthesized using 2′-O-thionocarbamate-protected nucleoside phosphoramidites. Methods are described in, e.g., Dellinger et al., J. American Chemical Society 133, 11540-11556 (2011); Threlfall et al., Organic & Biomolecular Chemistry 10, 746-754 (2012); and Dellinger et al., J. American Chemical Society 125, 940-950 (2003). Chemical modifications useful herein have been described for use in other Cas systems. See, e.g., US2018/0119140, which is incorporated herein by reference.

The modified crRNAs described herein can be used with any RNA-targeting CRISPR enzyme, such as the Cas13 family. The diverse Cas13 family contains at least four known subtypes, including Cas13a (formerly C2c2), Cas13b, Cas13c, and Cas13d. The Cas13 family is the only family of class 2 Cas enzymes known to exclusively target single-stranded RNA. Cas13 enzymes and systems are known in the art, see, e.g., U.S. Pat. No. 10,362,616, Abudayyeh, et al, C2c2 is a single-component programmable RNA-guided RNA-targeting CRISPR effector. Science 353, aaf5573 (2016); S. Shmakov, et al, Discovery and functional characterization of diverse class 2 CRISPR-Cas systems. Mol. Cell 60, 385-397 (2015); S. Shmakov, et al, Diversity and evolution of class 2 CRISPR-Cas systems. Nat. Rev. Microbiol. 15, 169-182 (2017). A. A. Smargon, et al, Cas13b is a type VI-B CRISPR-associated RNA-guided RNase differentially regulated by accessory proteins Csx27 and Csx28. Mol. Cell 65, 618-630.e7 (2017); J. S. Gootenberg, et al, Nucleic acid detection with CRISPR-Cas13a/C2c2. Science 356, 438-442 (2017); 0. 0. Abudayyeh, et al, RNA targeting with CRISPR-Cas13. Nature 550, 280-284 (2017). Each of these documents is incorporated herein. Such systems include the use of catalytically inactive enzymes, where useful.

In one embodiment, the Cas13 protein is Cas13a, Cas13b, Cas13c, or Cas13d. In one embodiment, the Cas13 comprises one or more mutations the HEPN domain(s).

The Cas13 protein may be from any organism. In one embodiment, the Cas13 effector protein is from an organism of a genus selected from: Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Campylobacter, and Lachnospira. In one embodiment, the Cas13 is derived from Leptotrichia shahii, Leptotrichia wadei, Listeria seeligeri, Clostridium aminophilum, Carnobacterium gallinarum, Paludibacter propionicigenes, or Listeria weihenstephanensis. Also provided herein are methods of enhancing the stability of a crRNA by modifying a crRNA with one or more chemically modified nucleotides as described herein. Methods for synthesizing chemically modified polynucleotides are known in the art, including commercially available services. For example, chemically modified polynucleotides are available from Synthego, Metabion, and IDT, amongst others.

Also provided herein is a method of enhancing modulation of gene transcripts in a cell. The method includes introducing into the cell a modified crRNA as described herein; and a Cas13 polypeptide, an mRNA encoding a Cas13 polypeptide, and/or a recombinant expression vector comprising a nucleotide sequence encoding a Cas13 polypeptide. The modified crRNA guides the Cas13 polypeptide to the target RNA sequence, whereby the modified crRNA induces regulation of the target RNA with an enhanced activity relative to a corresponding unmodified crRNA. Such enhanced activity may include increased stability of the modified crRNA and/or increased specificity of the modified crRNA for the target RNA. The term “increased stability” refers to modifications that stabilize the structure of any molecular component of the CRISPR system, in particular the crRNA. The term includes modifications that decrease, inhibit, diminish, or reduce the degradation of any molecular component of the CRISPR system. The term “increased specificity” refers to modifications that increase the specific activity (e.g., the on-target activity) of any molecular component of the CRISPR system, e.g., the crRNA. The term includes modifications that decrease, inhibit, diminish, or reduce the non-specific activity (e.g., the off-target activity) of any molecular component of the CRISPR system. The term “enhanced activity,” with respect to components of the CRISPR system and in the context of gene regulation, refers to an increase or improvement in the efficiency and/or the frequency of inducing, modulating, regulating, or controlling transcript modulation.

The modified crRNA as described herein and Cas13 polypeptide, mRNA encoding a Cas13 polypeptide, and/or recombinant expression vector comprising a nucleotide sequence encoding a Cas13 polypeptide variant or fragment thereof are introduced into the cell using any suitable method such as by electroporation. In certain instances, the modified crRNA is complexed with a Cas nuclease (e.g., Cas13 polypeptide) or a variant or fragment thereof to form a ribonucleoprotein (RNP)-based delivery system for introduction into a cell (e.g., an in vitro cell such as a primary cell for ex vivo therapy, or an in vivo cell such as in a patient). In other instances, the modified crRNA is introduced into a cell (e.g., an in vitro cell such as a primary cell for ex vivo therapy, or an in vivo cell such as in a patient) with an mRNA encoding a Cas nuclease (e.g., Cas13 polypeptide) or a variant or fragment thereof. In yet other instances, the modified crRNA is introduced into a cell (e.g., an in vitro cell such as a primary cell for ex vivo therapy, or an in vivo cell such as in a patient) with a recombinant expression vector comprising a nucleotide sequence encoding a Cas nuclease (e.g., Cas13 polypeptide) or a variant or fragment thereof.

To use the crRNA variants described herein, it may be desirable to provide them in conjunction with a nucleic acid that encodes a Cas13 protein. The nucleic acid encoding the Cas13 may be cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the Cas13 protein variant for production of the same. The nucleic acid encoding the Cas13 protein can also be cloned into an expression vector, for administration to a plant cell, animal cell, preferably a mammalian cell or a human cell, fungal cell, bacterial cell, or protozoan cell.

To obtain expression, a sequence encoding a Cas13 protein is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available.

Methods for introducing polypeptides and nucleic acids into a target cell (host cell) are known in the art, and any known method can be used to introduce a nuclease or a nucleic acid into a cell. Non-limiting examples of suitable methods include electroporation, viral or bacteriophage infection, transfection, conjugation, protoplast fusion, lipofection, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like. In one embodiment, the target RNA is mRNA and the modulation comprises inhibition, cutting, editing or modulation of protein expression of the target RNA. Target RNAs include, without limitation, mRNA, viral RNAs, noncoding RNAs, miRNA, piRNAs, circRNAs, synthetic RNA and other RNAs.

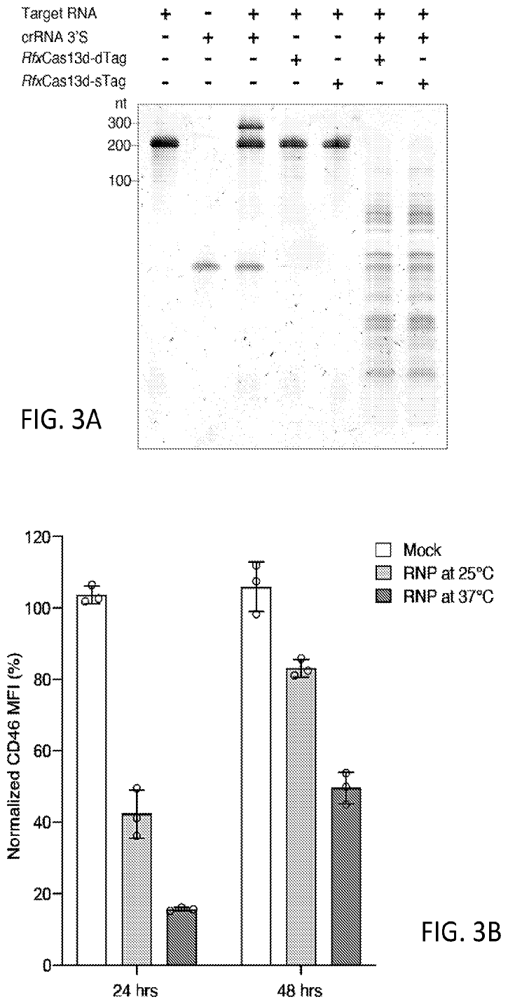

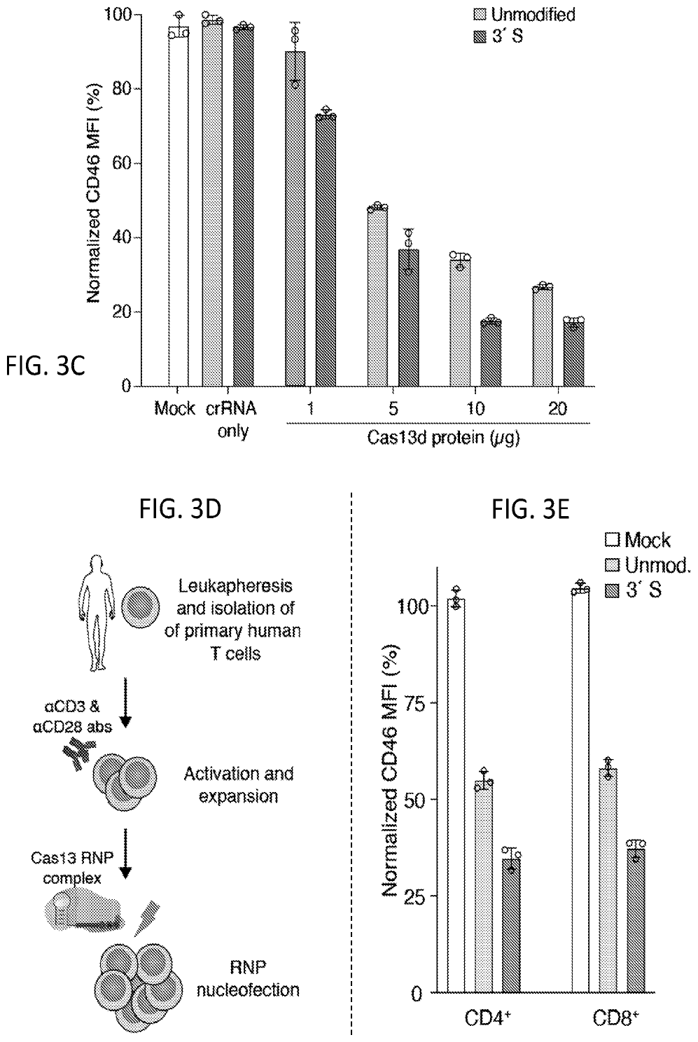

To functionally test the presence of the correct transcript modulation, the target RNA can be analyzed by standard methods known to those in the art. For example, denaturing gel electrophoresis can be performed to determine whether the target RNA has been fragmented (FIG. 3A). Other techniques, including FACS can be performed, using antibodies directed to the proteins corresponding to the target RNAs. See, e.g., Example 2. Other methods useful to test the presence of the correct transcript modulation include, without limitation, qPCR and RNA-seq.

In certain embodiments, the nuclease-mediated transcript-modulating efficiency of a target DNA sequence in a cell is enhanced by at least about 0.5-fold, 0.6-fold, 0.7-fold, 0.8-fold, 0.9-fold, 1-fold, 1.1-fold, 1.2-fold, 1.3-fold, 1.4-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 25-fold, 30-fold, 35-fold, 40-fold, 45-fold, 50-fold, or greater in the presence of a modified crRNA described herein compared to the corresponding unmodified crRNA sequence.

In another embodiment, a composition is provided which includes the modified crRNA as described herein, wherein the spacer sequence comprises a sequence complementary to the SARS-CoV-2 leader sequence. Also provided is a method of treating a COVID-19 infection in a subject in need thereof. The method includes introducing into a subject having, or suspected of having, COVID-19 infection, a composition which includes the modified crRNA as described herein, wherein the spacer sequence comprises a sequence complementary to the SARS-CoV-2 leader sequence and a Cas13 polypeptide, an mRNA encoding a Cas13 polypeptide, and/or a recombinant expression vector comprising a nucleotide sequence encoding a Cas13 polypeptide, wherein the modified crRNA guides the Cas13 polypeptide to the SARS-CoV-2 leader sequence, and wherein the modified crRNA induces knockdown of the SARS-CoV-2 virus, thereby treating COVID-19 infection. In one embodiment, the composition is administered as part of a mRNA vaccine together with an ex vivo delivery of Cas13 RNPs via a nucleofection/electroporation method.

The terms “subject,” “patient,” and “individual” are used herein interchangeably to include a human or animal. For example, the animal subject may be a mammal, a primate (e.g., a monkey), a livestock animal (e.g., a horse, a cow, a sheep, a pig, or a goat), a companion animal (e.g., a dog, a cat), a laboratory test animal (e.g., a mouse, a rat, a guinea pig, a bird), an animal of veterinary significance, or an animal of economic significance.

### Specific Embodiments

1. A modified crRNA comprising a spacer sequence and a direct repeat sequence, wherein the crRNA comprises one or more chemically modified nucleotides.
2. The modified crRNA of embodiment 1, wherein the spacer sequence comprises at least 23 nucleotides, and is complementary to a target RNA sequence.
3. The modified crRNA of embodiment 1, wherein the direct repeat sequence is 5′ to the spacer sequence.
4. The modified crRNA of any of embodiments 1 to 3, wherein the chemically modified nucleotides are located at the 5′ end of the crRNA or the 3′ end of the crRNA.
5. The modified crRNA of any of embodiments 1 to 4, comprising chemically modified nucleotides throughout the crRNA.
6. The modified crRNA of any one of embodiments 1 to 5, comprising 1, 2, 3, 4, or 5 modified nucleotides at the 3′ end of the spacer sequence.
7. The modified crRNA of any one of embodiments 1 to 5, comprising 1, 2, 3, 4, or 5 modified nucleotides located downstream of the 3′ end of the spacer sequence.
8. The modified crRNA of any of embodiments 1 to 7, wherein the chemically modified nucleotide comprises a modification is selected from 2′-O-methyl (M), 3′phosphorothioate (S), 2′O-methyl-3′-phosphorothioate (MS) and 3′ Inverted thymine (invT).
9. The modified crRNA of any of embodiments 1 to 8, wherein the chemical modification comprises a 2′O-methyl (M) modification.
10. The modified crRNA of any of embodiments 1 to 9, wherein the chemical modification comprises a 3′phosphorothioate (S) modification.
11. The modified crRNA of any of embodiments 1 to 10, wherein the chemical modification comprises a 2′O-methyl-3′-phosphorothioate (MS) modification.
12. The modified crRNA of any of embodiments 1 to 11, wherein the chemical modification comprises a 3′ inverted thymine (invT) modification.
13. A method of enhancing the stability of a crRNA, comprising modifying a crRNA comprising a spacer sequence and a direct repeat sequence, with one or more chemically modified nucleotides.
14. The method of embodiment 13, wherein the one or more chemically modified nucleotides comprises a chemical modification selected from 2′-O-methyl (M), 3′phosphorothioate (S), 2′O-methyl-3′-phosphorothioate (MS) and 3′ inverted thymine (invT).
15. The method of embodiment 13 or 14, wherein the spacer sequence comprises at least 23 nucleotides, and is complementary to a target RNA sequence.
16. The method of any one of embodiments 13 to 15, wherein the direct repeat sequence is 5′ to the spacer sequence.
17. The method of any one of embodiments 13 to 16, wherein the chemically modified nucleotides are located at the 5′ end of the crRNA or the 3′ end of the crRNA.
18. The method of any one of embodiments 13 to 17, comprising chemically modified nucleotides throughout the crRNA.
19. The method of any one of embodiments 13 to 17, comprising 1, 2, 3, 4, or 5 modified nucleotides at the 3′ end of the spacer sequence.
20. The method of any one of embodiments 13 to 17, comprising 1, 2, 3, 4, or 5 modified nucleotides located downstream of the 3′ end of the spacer sequence.
21. The method of any one of embodiments 13 to 20, wherein the chemically modified nucleotide comprises a modification is selected from 2′-O-methyl (M), 3′phosphorothioate (S), 2′O-methyl-3′-phosphorothioate (MS) and 3′ Inverted thymine (invT).
22. The method of any one of embodiments 13 to 21, wherein the chemical modification comprises a 2′O-methyl (M) modification.
23. The method of any one of embodiments 13 to 22, wherein the chemical modification comprises a 3′phosphorothioate (S) modification.
24. The method of any one of embodiments 13 to 23, wherein the chemical modification comprises a 2′O-methyl-3′-phosphorothioate (MS) modification.
25. The method of any one of embodiments 13 to 24, wherein the chemical modification comprises a 3′ Inverted thymine (invT) modification.
26. A method of enhancing modulation of gene transcripts in a cell, the method comprising: introducing into the cell
   (a) the modified crRNA of any of embodiments 1 to 12; and
   (b) a Cas13 polypeptide, an mRNA encoding a Cas13 polypeptide, and/or a recombinant expression vector comprising a nucleotide sequence encoding a Cas13 polypeptide,
   wherein the modified crRNA guides the Cas13 polypeptide to the target RNA sequence, and wherein the modified crRNA induces regulation of the target RNA with an enhanced activity relative to a corresponding unmodified crRNA.
27. The method of embodiment 26, wherein the enhanced activity comprises increased stability of the modified crRNA and/or increased specificity of the modified crRNA for the target RNA.
28. The method of embodiment 26 or 27, wherein the target RNA is mRNA and the modulation comprisesdownregulation of protein expression of the target mRNA.
29. A composition comprising the modified crRNA of any one of embodiments 1 to 12, wherein the spacer sequence comprises a sequence complementary to the SARS-CoV-2 leader sequence.
30. A method of treating COVID-19 infection, the method comprising introducing into a subject having, or suspected of having, COVID-19 infection:
   (a) the composition according to embodiment 29; and
   (b) a Cas13 polypeptide, an mRNA encoding a Cas13 polypeptide, and/or a recombinant expression vector comprising a nucleotide sequence encoding a Cas13 polypeptide,
   wherein the modified crRNA guides the Cas13 polypeptide to the SARS-CoV-2 leader sequence, and wherein the modified crRNA induces knockdown of the SARS-CoV-2 virus, thereby treating COVID-19 infection.
31. A method of modulating gene transcripts in a primary cell, the method comprising: introducing into the cell
   (a) the modified crRNA of any of embodiments 1 to 12; and
   (b) a Cas13 polypeptide, an mRNA encoding a Cas13 polypeptide, and/or a recombinant expression vector comprising a nucleotide sequence encoding a Cas13 polypeptide,
   wherein the modified crRNA guides the Cas13 polypeptide to the target RNA sequence, and wherein the modified crRNA induces regulation of the target RNA with an enhanced activity relative to a corresponding unmodified crRNA.
32. The method of embodiment 31, wherein the primary cell is cultured in vitro for use in ex vivo therapy.
33. The method of embodiment 31, wherein the primary cell is contacted in vivo with the modified crRNA of (a) and the Cas13 polypeptide, an mRNA encoding a Cas13 polypeptide, and/or a recombinant expression vector comprising a nucleotide sequence encoding a Cas13 polypeptide of (b).
34. The method of any of embodiments 31 to 33, wherein the primary cell is a stem cell or an immune cell.
35. The method of embodiment 34, wherein the primary cell is a T-cell.
36. The method of embodiment 35, wherein the T-cell is a CD4+ or CD8+.

The following examples are illustrative only and are not intended to limit the present invention.

### EXAMPLES

### Example 1: Modified crRNAs

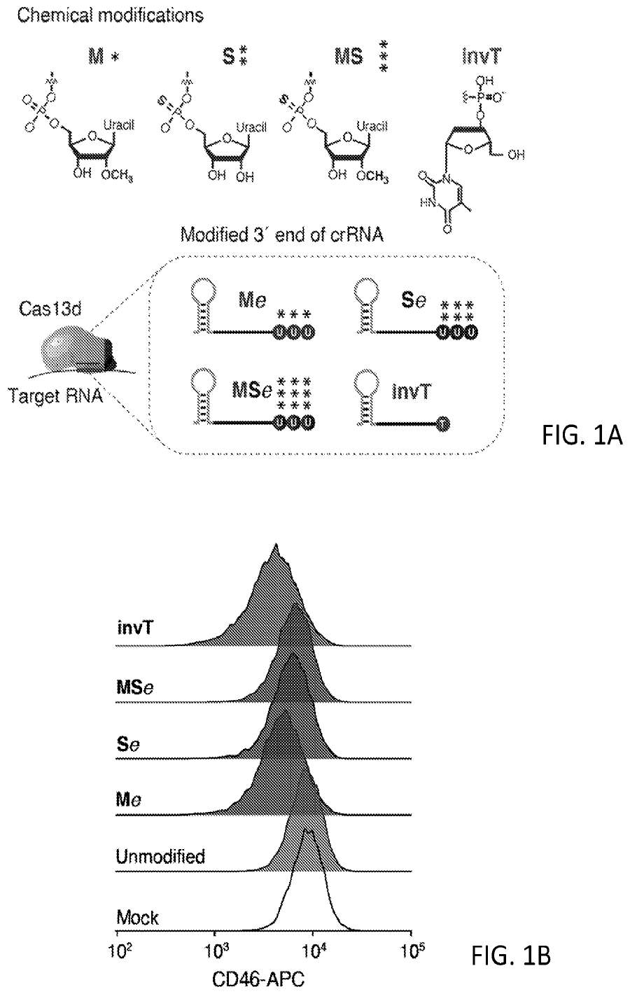

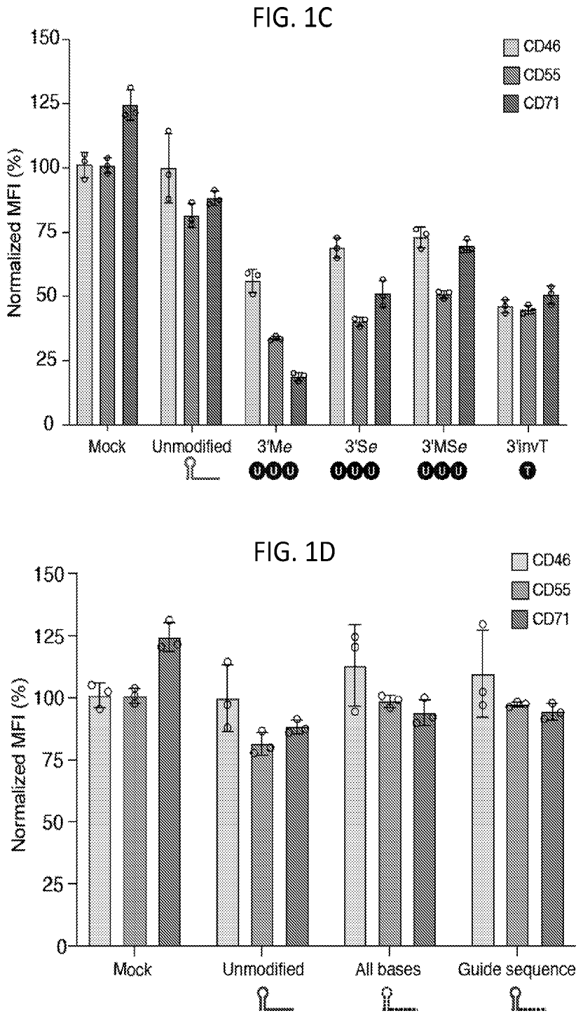

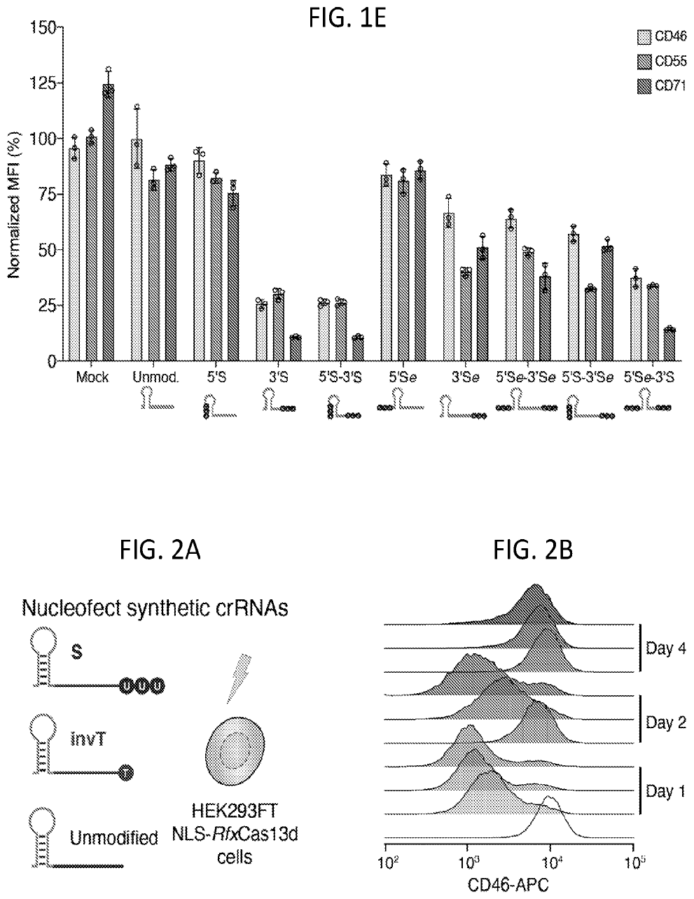

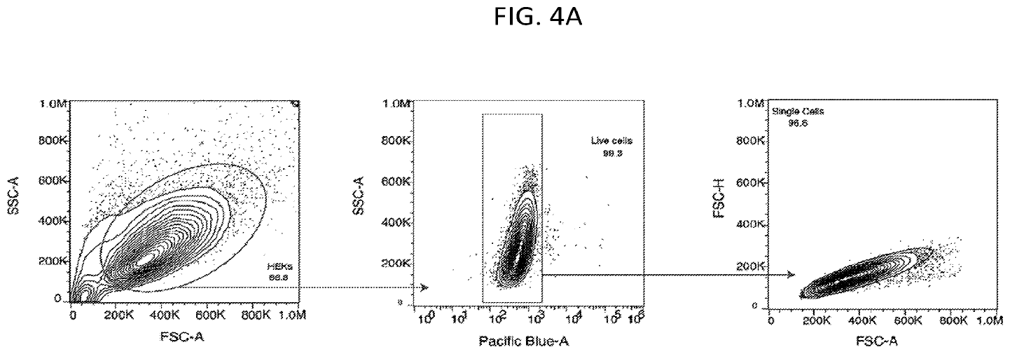

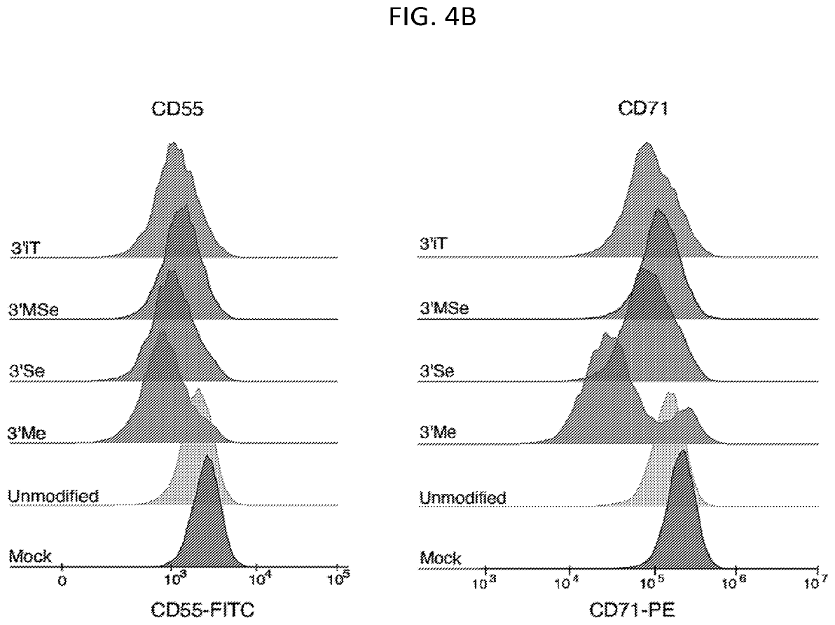

First, we assessed the degree of target RNA knockdown efficiency upon exogenous delivery of unmodified and chemically-modified crRNAs. We synthesized crRNAs with different chemical modifications at three uridine nucleotides (3xU) at the 3′ end of a 23 nt spacer sequence or with an inverted thymidine capping the 3′ end (FIG. 1A). We tested 3 different modifications of the 3xU bases which have been reported before to improve RNA stability and evade secondary immune responses^{15,18,19}: 2′-O-methylation (M), phosphorothioate linkage (S), and 2′-O-methylation and phosphorothioate linkage (MS). Although all of these modified crRNAs add extra bases at the 3′ end, we previously demonstrated that nucleotide mismatches to the target site beyond the 23 nt RNA-RNA hybridization interface do not interfere with target knockdown efficiency¹⁷. We chose to assess target knockdown efficiency of three broadly expressed cell surface proteins (CD46, CD55, CD71) that can be efficiently targeted with RfxCas13d17. CrRNAs The synthesized crRNAs. The synthesized crRNAs were nucleofected into monoclonal RfCas13d-NLS expressing HEK293FT cells and after three days cells were analyzed for protein expression by fluorescent activated cell sorting (FACS). Importantly, cells needed to be pre-treated with doxycycline to induce Cas13 expression prior to crRNA delivery, likely because Cas13 binding to crRNAs can protect crRNAs against endogenous RNA nuclease mediated decay. For unmodified crRNAs, we noticed that target knockdown for each of the three target transcripts was barely detectable relative to non-targeting crRNAs, suggesting that indeed unmodified crRNAs get rapidly cleared in human cells and cannot yield lasting knockdown effects (FIG. 1B, FIG. 1C and FIG. 4B). On the other hand, all chemically modified crRNAs improved target knockdown but did so to varying degrees (one-way ANOVA, p<10⁻⁴). We found that M-modified crRNAs had the overall largest knockdown (80% knockdown of CD71) but there were greater variability among target transcripts. In contrast, we found that the InvT modification improved knockdown efficiency in a more consistent manner for the three cell surface markers (55%) (FIG. 1c). Surprisingly, the combination of M and S modifications did not result in improved knockdown than either individual modification (M or S) across all 3 target transcripts (one-way ANOVA, Tukey-corrected p=0.14 [M], p=0.89 [S]).

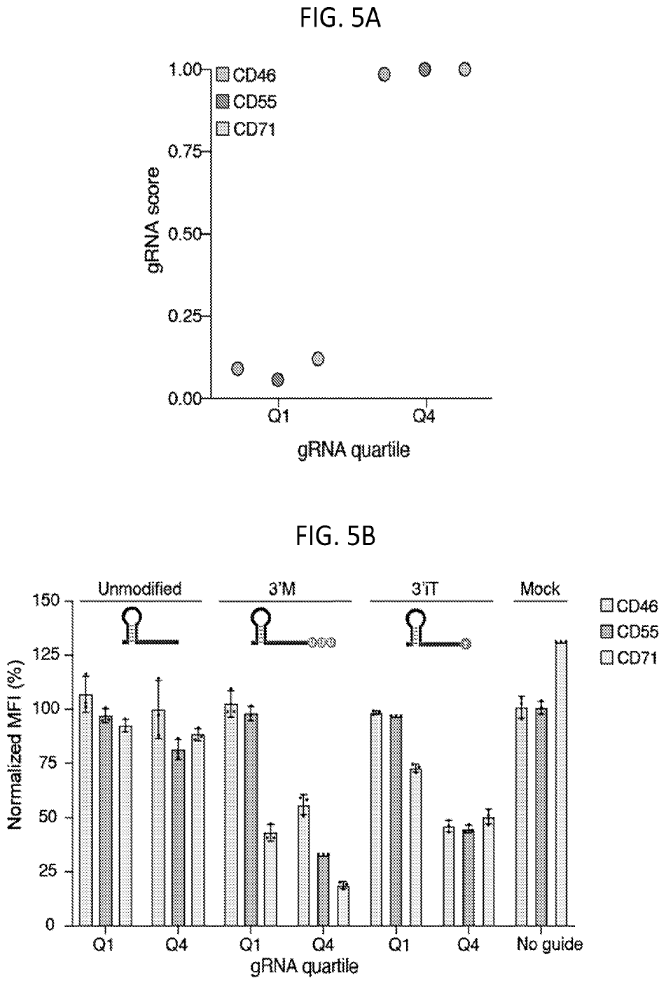

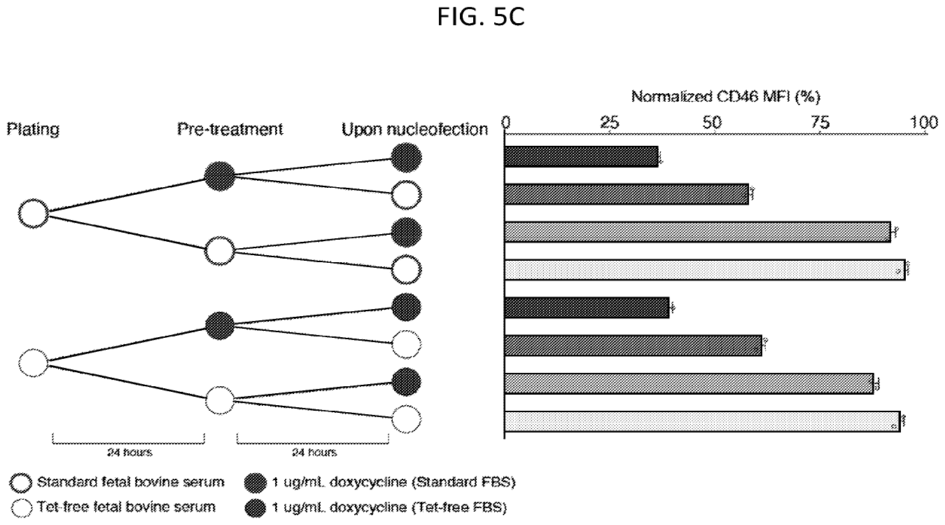

In addition to improving Cas13-mediated target knockdown, we wondered if chemical modifications can close the gap between low efficiency gRNAs and high efficiency gRNAs. Previously used a high-throughput screen to identify optimal Cas13 guide sequence parameters and developed a scoring metric to predict crRNA efficiency¹⁷. We chose one low-scoring guide sequence per target gene in addition to the high-scoring guide sequence used above (FIG. 5A) and found that chemically modified crRNAs do not generally improve activity of low scoring guide sequences (FIG. 5B). This suggests that chemical modification may generally improve crRNA stability but does not improve gRNA efficiency. Stability of the crRNA is a key element of efficient Cas13 knockdown. Using M-modified crRNAs, we found that substantial knockdown was only possible when Cas13 was already expressed (e.g. starting at 24 hours before nucleofection or at the time of cell plating) (FIG. 5C).

Encouraged by these results, we next examined whether alternative placement of modified bases could further improve crRNA stability and transcript knockdown. Specifically, we tested if more extensively M-modified crRNAs could further increase crRNA stability without interfering with target knockdown, as previously shown for DNA-targeting Cas9 sgRNA²⁰. We synthesized crRNAs containing M-modifications along all the bases in the crRNA (53 modified bases) or all bases in the spacer sequence (23 modified bases). For all three targeted genes, the more extensive M-modifications abrogated Cas13 knockdown compared to unmodified crRNAs, presumably by disrupting the Cas13-crRNA interaction (FIG. 1D). Since partial modifications at the 3′ end boost knockdown, we decided to more systematically test the effect of partial modifications on the crRNA 5′ and/or 3′ends. We tested crRNAs with 3′phosphorothioate (S) modification at the first and last three nucleotides of the crRNA's 5′ and 3′ ends (5S and 3S, respectively) or using a 3xU 5′ or 3′ extension (5′Se and 3′Se, respectively) similar to shown in FIG. 1a. We also tested all combinations of 5′ and 3′ end modifications. We found marked improvement of target knockdown for crRNAs with phosphorothioate modifications at the 3′ end of the spacer sequence (FIG. 1E). Interestingly, 3′ phosphorothioate modifications improved knockdown efficiency to a greater degree when placed within the spacer sequence (3'S) compared to being placed in 3xU extensions (3′Se). Modifications at the 5′ end of the crRNA alone, or in conjunction with 3′ modifications did not improve target knockdown efficiency (one-way ANOVA, Tukey-corrected p=0.99 for all comparisons with added 5′ modifications). These results suggest that exonucleases or degradation processes at the crRNA 3′ end are a major hurdle for increases Cas13 activity using synthetic crRNAs²¹.

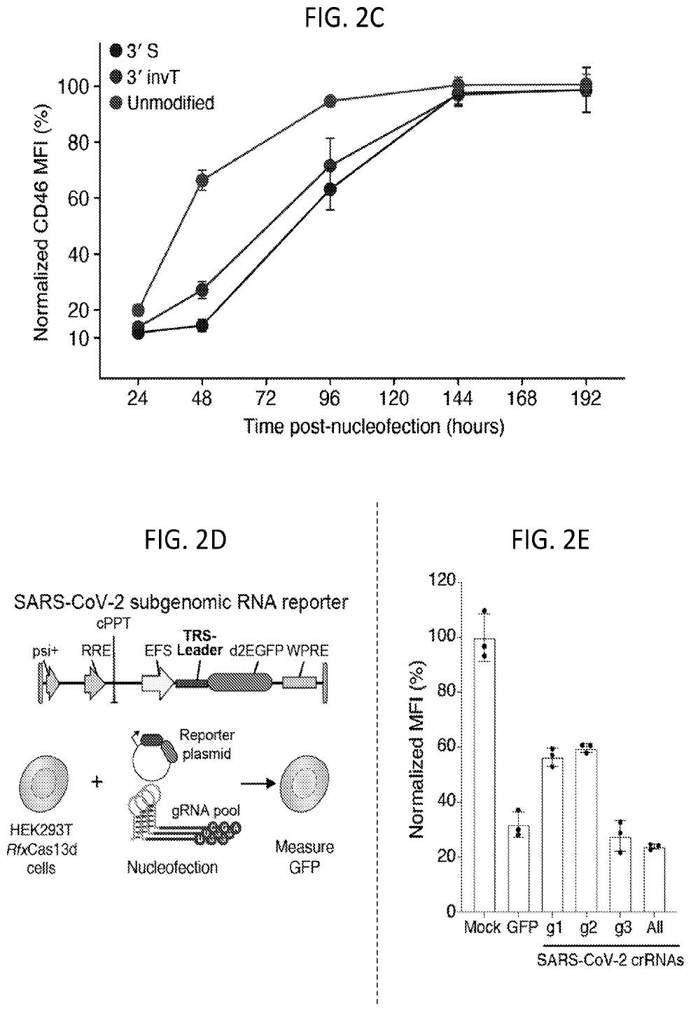

Next, we sought to assess the temporal dynamics of Cas13 activity with synthetic crRNAs by comparing knockdown of CD46 over time for 3′ nvT, 3'S and unmodified crRNAs relative to non-targeting crRNAs (FIG. 2A). All three targeting crRNAs, including unmodified crRNAs, yielded almost complete CD46 knockdown at 24 hours after nucleofection with around 95% protein loss for 3'S-modified crRNAs (FIG. 2B). While CD46 expression quickly recovered in cells targeted with unmodified crRNAs, 3'S crRNAs led to pronounced knockdown 48 hrs after crRNA delivery (FIG. 2C). Even 4 days after nucleofection the modified crRNAs resulted in ˜40% knockdown. Both crRNA modifications extend knockdown effects by about 2 days compared to unmodified crRNAs.

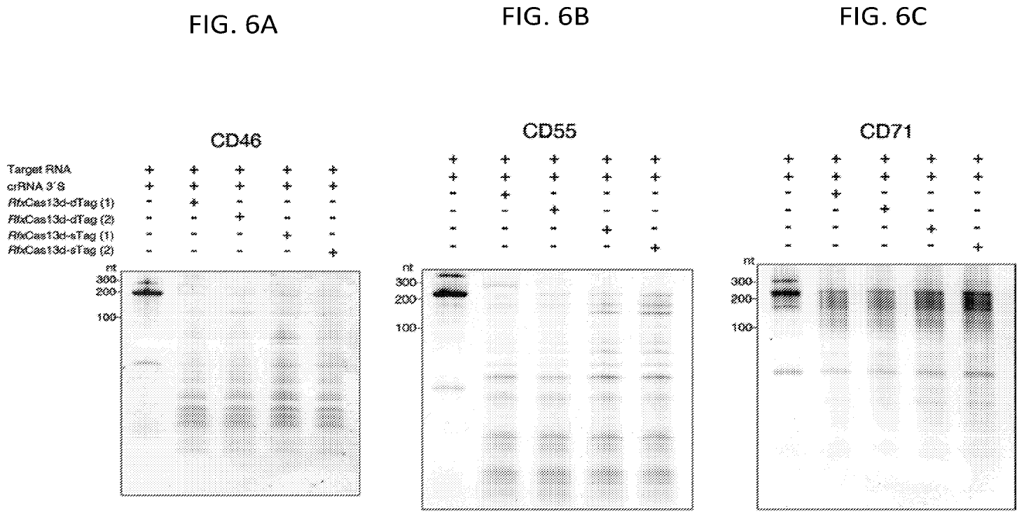

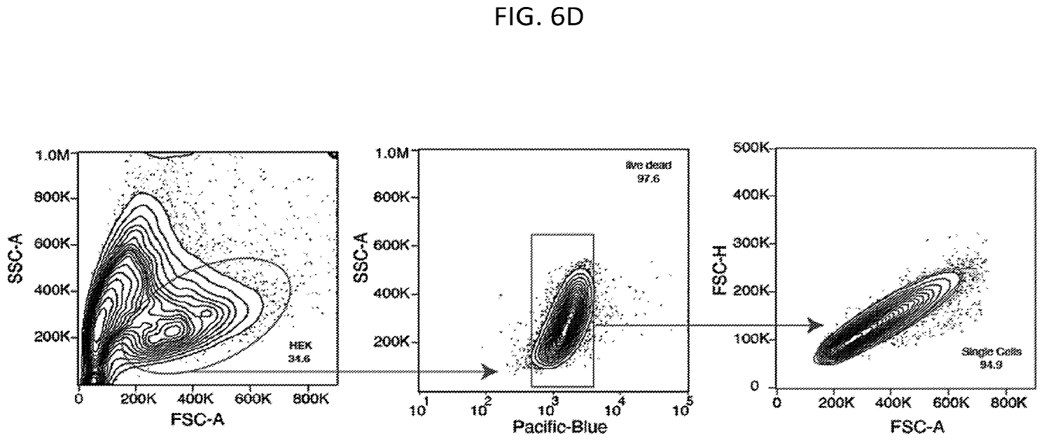

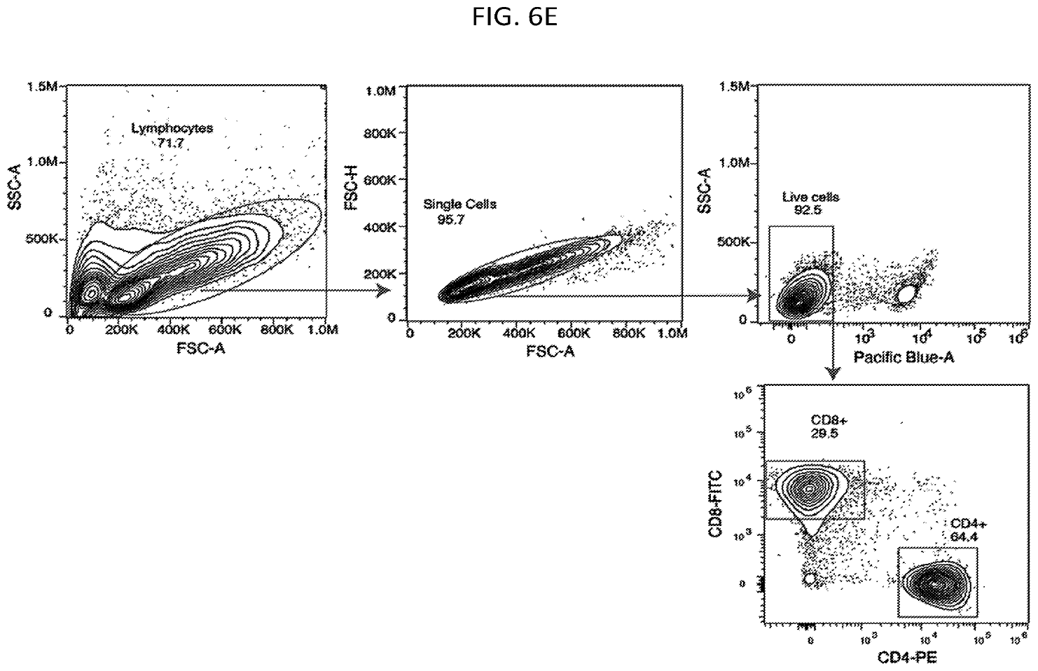

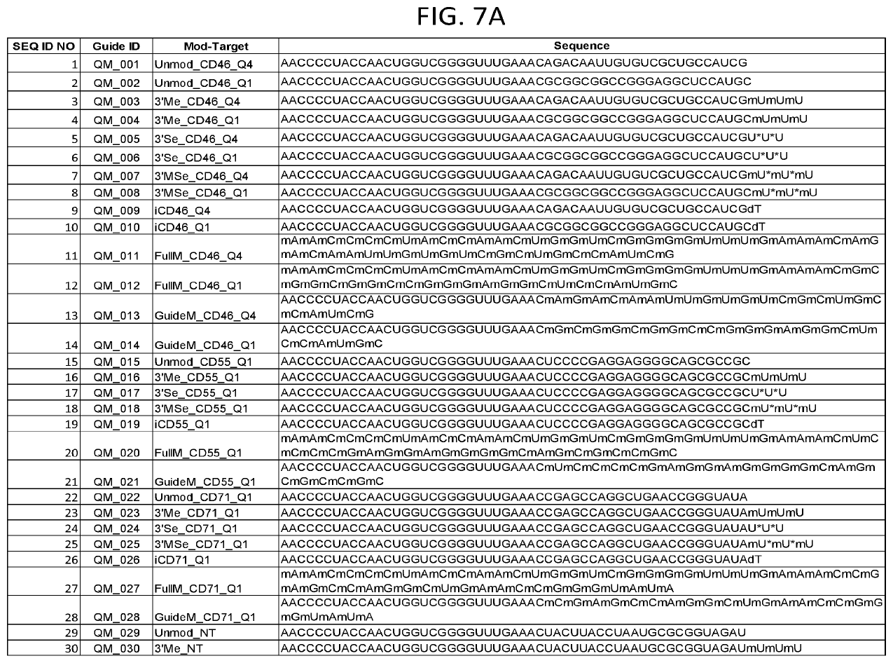

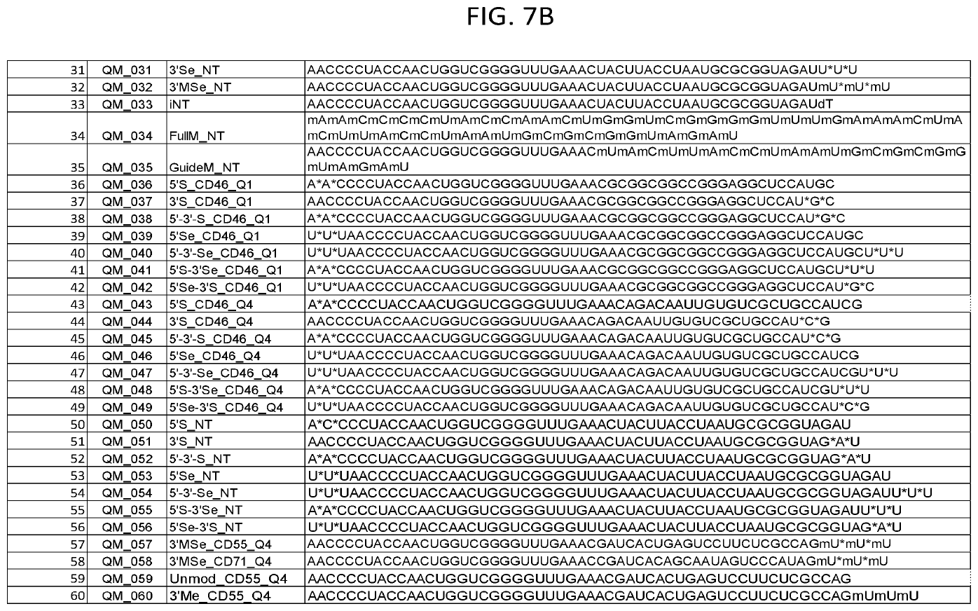

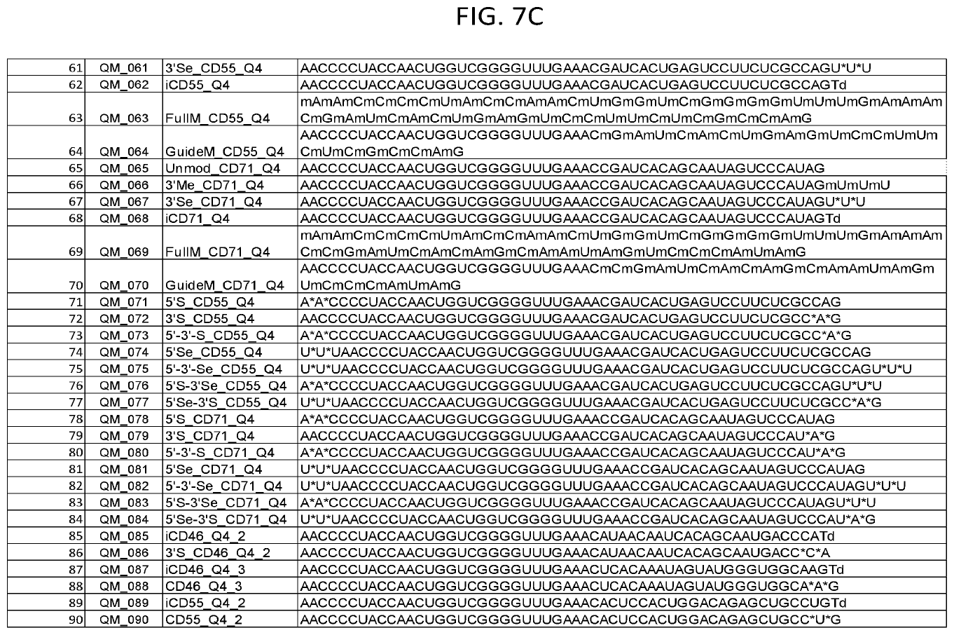

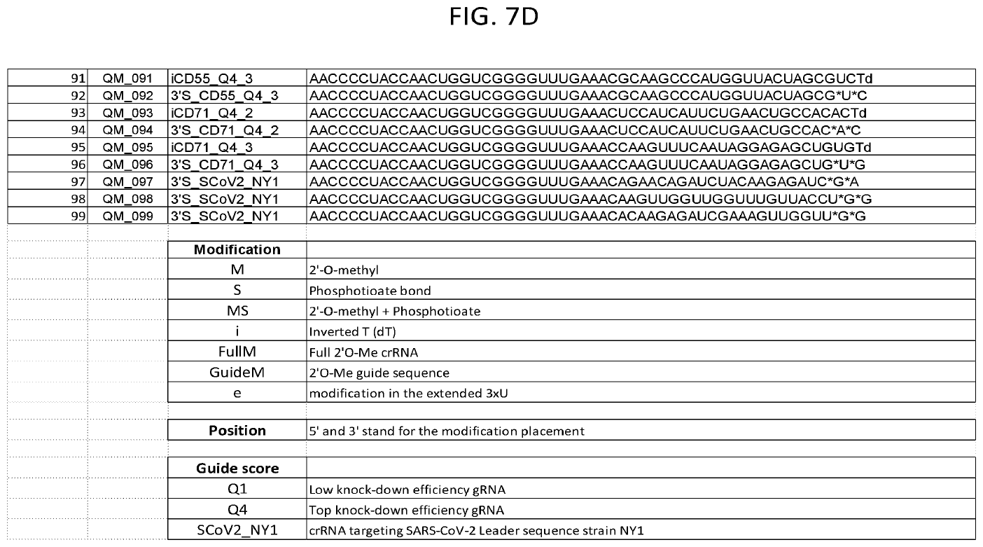

In addition to delivery of chemically-modified crRNAs for extended knockdown, another major challenge is delivery of the effector protein into systems that cannot easily be genetically modified to continuously express Cas13. Therefore, we next sought to evaluate if we could preassemble Cas13 RNP complexes with synthetic crRNAs and deliver these RNPs into primary human cells. First, we confirmed that preassembled RNPs with recombinant RfxCas13d-NLS (rCas13d) could efficiently degrade different target RNAs in vitro (FIG. 3A and FIG. 6A-6C). Moreover, we compared if different protein purification methods would alter Cas13 activity in vitro and found that rRfxCas13d-NLS-1 (heparin fractions pool) performed consistently well across all targets, (FIG. 3B-D). We found that purified rCas13d with or without an N-terminal MKIEE expression and solubility tag resulted in similar nuclease activity in vitro. We proceeded to test the preassembled rCas13d RNP complexes in human HEK293FT cells and found that greater knockdown is achieved by preassembling RNPs at 37° C. instead of 25° C. (FIG. 3B, FIG. 6D). The RNP complexes resulted in 85% target knockdown for CD46 at 24 hours post-nucleofection. In order to identify the optimal conditions for RNP knockdown, we nucleofected RNPs in HEK293FT cells using different protein amounts, holding the molar ratio of rCas13d to crRNA (1.2) constant. We found that 10 μg of rCas13d protein leads to the strongest knockdown and that additional rCas13d does not lead to greater knockdown (two-tailed t-test, p=0.7) (FIG. 3C). As before, we find that using chemically-modified crRNAs improves RNA knockdown (two-tailed paired t-test, p=5×10³).

Using these optimized conditions for RNP formation and nucleofection, we isolated human T-cells from healthy donors, stimulated the cells with anti-CD3 and anti-CD28 antibodies, and asked whether we could knockdown CD46 expression in primary T-cells using these RNP complexes (FIG. 3D). We assessed CD46 expression in CD4+ and CD8+ populations separately 24 hours after nucleofecting activated T-cells (FIG. 3E and FIG. 6E). For both T-cell populations we achieved a 60-65% knockdown of CD46 with 3'S modified crRNAs (FIG. 3F). With unmodified crRNAs, we achieved only 40-45% knockdown of CD46. Taken together, these experiments demonstrate that Cas13 RNPs can modify gene expression in primary immune cells and that modified crRNAs lead to greater gene knockdown than unmodified crRNAs.

Targeting RNA has become more relevant in recent years due to the rise of global pandemics driven by new viruses. Currently, the world is faced with a pandemic of novel coronavirus disease 2019 (COVID-19) and till now there are no proven preventative treatment or vaccine. The causing severe acute respiratory syndrome coronavirus 2 (SARS-CoV-2) belongs to a family of positive-sense RNA viruses, which open the possibility to use an alternative antiviral approach using CRISPR-Cas13-based strategy together with synthetic crRNAs with the goal of recognizing and degrading intracellular viral genome, as previously suggested^{12,23}.

Recently, several groups have suggested the possibility of using Cas13 to target severe acute respiratory syndrome coronavirus 2 (SARS-CoV-2), a positive-sense RNA virus responsible for the current global pandemic of novel coronavirus disease 2019 (COVID-19)^{21,23}. To test if synthetic crRNA can degrade SARS-CoV-2, we designed a reporter construct that contains the SARS-CoV-2 leader sequence in the 5′UTR of a GFP-reporter gene (FIG. 3F). The SARS-CoV-2 leader sequence is a component of all SARS-CoV-2 subgenomic RNAs and represents a universal targeting site for all subgenomic viral transcripts^{24,25}. We used our Cas13 gRNA design software to design three gRNAs with high predicted target knockdown efficacy. We cloned these gRNAs into an all-in-one construct expressing cytosolic Cas13d, transfected ACE2-expressing HeLa cells and infected these cells with SARS-CoV-2 at a MOI of 0.1. Cells transfected with leader sequence targeting gRNAs showed significant reduction of viral N protein 24 hours post infection (FIG. 2D). Similar to experiments with endogenous human transcripts, we found that 3'S-modified crRNA targeting the SARS-CoV-2 leader sequence can suppress protein expression, despite targeting an untranslated sequence (FIG. 2E). These results suggest that Cas13 together with chemically modified crRNAs may represent an efficient and programmable therapeutic approach to target universal SARS-CoV-2 sequences.

### Example 2: Methods

Biochemical in vitro RNA cleavage assays.

200 bp targets for CD46, CD55 and CD71 were synthesized by preparative PCR amplification from total cDNA from HEK293FT cells, using primers with a T7 promoter sequence, transcripts where then purified using HiScribe™ T7 High Yield RNA Synthesis Kit (NEB). Purified RfxCas13d [NLS/NES] protein and synthetic crRNAs were mixed at 2:1 molar ratio in RNA Cleavage Buffer (25 mM Tris pH 7.5, 1 mM DTT, 6 mM MgCl2). The reaction was prepared on ice and incubated at 37° C. for 15 minutes prior to the addition of target at 1:2 molar ratio relative to RfxCas13d. The reaction was subsequently incubated at 37° C. for 45 minutes and quenched with 1 mL of enzyme stop solution (10 mg/mL Proteinase K, 4M Urea, 80 mM EDTA, 20 mM Tris pH 8.0) at 37° C. for 15 minutes. The reaction was then denatured with RNA Gel Loading Dye (ThermoFisher Scientific), gels were stained with SYBR Gold prior to imaging via Gel Doc EZ system (Bio-Rad).

We mixed the indicated components in a total reaction volume 10 ul, then incubated at 37° C. for 15 min, and put on ice to stop the reaction. In each reaction, the concentration of recombinant Cas13d (rCas13d) was 45 nM. We visualized the enzymatic cleavage by loading the entire reaction onto 1 lane of a 10% TBE-Urea gel. Gels were electrophoresed at 180 V for 35 min and then stained for 1 min in SYBR Gold. Gels are imaged on a BioRad GelDoc imager. (FIG. 3A).

We electroporated 100,000 human HEK293FT cells using recombinant Cas13d and a modified (3'S) or unmodified guide RNA targeting the human CD46 transcript. We electroporated 10 μg of rCas13d and 55 uM of guide RNA using a Lonza Nucleofector 4D. Prior to electroporation, we complexed the rCas13d as guide RNA for 15 minutes at either room temperature (25C) or 37C. We measured CD46 expression using a CD46-APC antibody after 24 or 48 hours. FIG. 4B.

Plasmids.

We generated RfxCas13d proteins from cloning the effector protein RfxCas13d [NLS/NES] was either a nuclear localization signal or nuclear export signal into TetO-[Cas13]-WPRE-EFS-rtTA3-2A-Blast. To test the SARS-CoV-2 knockdown, we introduced the ˜75 nt SARS-CoV-2 Leader sequence into the EFS-EGFPd2PEST-2A-Hygro (Addgene) plasmid right before the 5′UTR region of the ′NLS-d2EGFP reporter construct.

Cell Culture and Nucleofection.

HEK293FT (ATCC) and T cells were cultured at 37° C., 5% C02, and ambient oxygen levels. HEK293FT cells were maintained in DMEM (Caisson Labs) supplemented with 10% bovine growth serum. For all nucleofection experiments on HEK293FT-RfxCas13d cells, we seeded three replicates of ˜2M of cells with lug/mL of doxycycline 24 hours before nucleofection, using 1×10⁵ cells per nucleofection condition. HEK293FT cells were nucleofected with 3 μl of 2.5 μM synthetic crRNAs in 20 μl reaction of SF Cell Line Nucleofector Solution (Lonza V4XC-2032) using the Lonza Nucleofector 2b (program CM-130).

T cells were procured from a de-identified healthy donor's LeukoPaks (New York Blood Center). First, peripheral blood mononuclear cells (PBMCs) were isolated using Lymphoprep gradient centrifugation (StemCell Technologies 07811). CD8+ T cells were isolated from PBMCs by positive magnetic selection using EasySep Human CD8 Positive Selection Kit (StemCell Technologies 17853). CD4+ T cells were isolated from PBMCs by negative magnetic selection using EasySep Human CD4 T cell Isolation Kit (StemCell Technologies #7952).

Isolated T cells were then plated in ImmunoCult-XF T Cell Expansion Medium (StemCell Technologies 10981) supplemented with 10 ng μL⁻¹ recombinant human IL-2 (StemCell Technologies 78036) and activated with the ImmunoCult Human CD3/CD28 T Cell Activator (StemCell Technologies 10971).

Chemically Modified CRISPR RNA (crRNA) Synthesis

The crRNAs were synthesized using solid-phase phosphoramidite chemistry (Synthego CRISPRevolution platform). Following synthesis, postprocessing and purification steps, we quantified each crRNA by UV absorption using Nanodrop (ThermoScientific). We confirmed their identities and quality using an Agilent 1290 Infinity II high-performance liquid chromatography (HPLC) system coupled with an Agilent 6530B Quadrupole time of-flight mass spectrometry (Agilent Technologies) in negative ion polarity mode.

Recombinant Cas13d Production and Purification

Recombinant RfxCas13d proteins were expressed in E. coli NiCo2l(DE3) (C2529, NEB). Cells were grown in 1L of LB medium containing 40 mg/mL kanamycin with shaking at 30° C. Protein expression was induced by the addition of 0.4 mM IPTG and the temperature was reduced to 16° C. for 16 hours. Pelleted cells were disrupted by sonication. Proteins were purified from the lysate supernatant using HiTrapDEAEFF Sepharose, HisTrap HP and HiTrap HeparinHP columns on an Akta Go instrument (Cytiva). To produce sTag RfxCas13d, the N-terminal His-MPB fusion partner was cleaved with Sumo protease and removed. Purified RfxCas13d proteins were stored in 20 mM Tris-HCl, pH7.5, 500 mM NaCl, 1 mM EDTA, 1 mM DTT and 50% glycerol (v/v). Protein concentration was determined by Bradford assay (Bio-Rad).

Nucleofection of crRNAs and Ribonucleoprotein (RNP) Complexes

For nucleofection experiments in HEK293FT-RfxCas13d cells, we seeded three replicates of ˜2×10⁶ cells with 1 ug/mL of doxycycline 24 hours before nucleofection, using 1×10⁵ cells per nucleofection condition. HEK293FT-RfxCas13d cells were nucleofected with 225 μmol of synthetic crRNAs in 20 μl reaction of SF Cell Line Nucleofector Solution (Lonza V4XC-2032) using the Lonza Nucleofector 4D (program CM-130). Immediately after nucleofection, cells were plated in pre-equilibrated D10 media (equilibrated to 37° C. and 5% CO₂) and remained there until flow cytometry analysis. For SARS-CoV-2 RNA knockdown, we nucleofected HEK293FT-RfxCas13d with 0.4 μg of TRS-Leader-SARS-CoV-2-d2eGFP plasmid together with 225 μmol of synthetic crRNAs and performed flow cytometry 24 hours after nucleofection. For RNP delivery experiments, purified RfxCas13d protein and crRNAs were assembled at a 1:2 molar ratio (Cas13 protein: crRNA) under two different RNP complexing conditions: 1) 10 mins at room temperature with nuclease reaction buffer (GenScript) and 2) 15 mins at 37° C. with RNA cleavage buffer (25 mM Tris pH 7.5, 1 mM DTT, 6 mM MgCl₂). After complexing the RfxCas13d RNPs were nucleofected into HEK293FT cells or primary T cells using 105 to 10⁶ cells in 20 μl of nucleofection solutions specific for each cell type. For HEK293FT nucleofection, cells were nucleofected in 20 μl reaction of SF Cell Line Nucleofector Solution (Lonza V4XC-2032) using the Lonza Nucleofector 4D (program CM-130). Immediately after nucleofection, HEK293FT cells were plated in pre-equilibrated D10 media (equilibrated to 37° C. and 5% CO₂) and remained there until flow cytometry analysis. For T cells nucleofection, two days after activation CD4+ and CD8+ T cells were combined, washed 2X in Dulbecco's PBS without calcium or magnesium (D-PBS, Caisson Labs) and resuspended in 20 μl P3 Primary Cell Nucleofector Solution (Lonza V4XP-3032). T cells were then nucleofected using the Lonza Nucleofector 4D (program E0-115). Nucleofections were performed in triplicate. Immediately after nucleofection T cells were plated in pre-equilibrated ImmunoCult-XF T Cell Expansion Medium supplemented with 10 ng μL⁻¹ recombinant human IL-2 (equilibrated to 37° C. and 5% C02). Before flow cytometry analysis, nucleofected T cells were washed with D-PBS and stained with LIVE/DEAD Fixable Violet Dead Cell Stain (ThermoFisher) for 5 minutes at room temperature in the dark before proceeding with antibody stain.

Biochemical in vitro RNA cleavage assays We synthesized 200 bp target RNAs for CD46, CD55 and CD71 by PCR amplification of templates from total cDNA from HEK293FT cells and then performed T7 in vitro RNA transcription. To prepare the cDNA library, we extracted total RNA from HEK293FT cells using Direct-zol purification (Zymo) and reverse transcribed 1 ug of RNA into cDNA using Revert-Aid (ThermoFisher). We amplified 200 bp amplicons from the cDNA using primers that include a T7 promoter sequence on the 5′ end (Table 2). We performed in vitro RNA transcription using the HiScribe T7 High Yield RNA Synthesis Kit (NEB). After transcription, RNA was purified using Monarch RNA Cleanup Kit (NEB) and quantified by Nanodrop spectrophotometer (ThermoFisher).

Purified RfxCas13d proteins and synthetic crRNAs were mixed (unless otherwise indicated) at 2:1 molar ratio in RNA Cleavage Buffer (25 mM Tris pH 7.5, 1 mM DTT, 6 mM MgCl₂). The reaction was prepared on ice and incubated at 37° C. for 15 minutes prior to the addition of target RNA at 1:2 molar ratio relative to RfxCas13d protein. The reaction was incubated at 37° C. for 45 minutes and quenched with 1 μL of enzyme stop solution (10 mg/mL Proteinase K, 4M Urea, 80 mM EDTA, 20 mM Tris pH 8.0) at 37° C. for 15 minutes. The samples were denatured at 95° C. for 5 minutes in 2X RNA Gel Loading Dye (ThermoFisher) and loaded onto 10% TBE-Urea gels (ThermoFisher). The gels were run at 180 V for 35 mins. After separation, gels were stained with SYBR Gold (ThermoFisher) prior to imaging via Gel Doc EZ system (Bio-Rad).

Flow Cytometry and Fluorescence-Activated Cell Sorting (FACS).

After nucleofection experiments HEK293FT-RfxCas13d [NLS/NES] and T cells were stained with the following antibodies: PE anti-human CD4 (clone RPA-T4, Biolegend 300507), FITC anti-human CD8a (clone RPA-T8, Biolegend 301006) and APC anti-human CD46 (clone TRA-2-10, Biolegend 352405). Antibody staining was performed for 20 minutes on ice. After staining the cells were washed 2X in D-PBS and acquired on Sony SH800S Cell Sorter. A minimum of 10,000 viable events was collected per sample.

Data Analysis

Data analysis and statistical testing was performed using GraphPad Prism 8 (GraphPad Software Inc.). Flow cytometry analysis and figure generation was performed using FlowJo v10 (BD). Specific statistical analysis methods are described in the figure legends where results are presented. Values were considered statistically significant forp values below 0.05.

All publications cited in this specification are incorporated herein by reference. While the invention has been described with reference to particular embodiments, it will be appreciated that modifications can be made without departing from the spirit of the invention. Such modifications are intended to fall within the scope of the appended embodiments.

### REFERENCES

1. Smargon, A. A., Shi, Y. J. & Yeo, G. W. Metagenomic Discovery To Transcriptomic Engineering. Nat. Cell Biol. 22, (2020).
2. Konermann, S. et al. Transcriptome Engineering with RNA-Targeting Type VI-D CRISPR Effectors. Cell 173, 665-676.e14 (2018).
3. Abudayyeh, 0. 0. et al. RNA targeting with CRISPR-Cas13. Nature 550, 280-284 (2017).
4. Cox, D, B. T. et al. RNA editing with CRISPR-Cas13 David. Science (80-.). 0180, 1-15 (2017).
5. Li, J. et al. Targeted mRNA demethylation using an engineered dCas13b-ALKBH5 fusion protein. Nucleic Acids Res. 48, 5684-5694 (2020).
6. Yang, L. Z. et al. Dynamic Imaging of RNA in Living Cells by CRISPR-Cas13 Systems. Mol. Cell 76, 981-997.e7 (2019).
7. Wang, H. et al. CRISPR-mediated live imaging of genome editing and transcription. Science (80-.). 2-6 (2019).
8. Gootenberg, J. S. et al. Multiplexed and portable nucleic acid detection platform with Cas13, Casl2a and Csm6. Science (80-.). 360, 439-444 (2018).
9. Gootenberg, J. S. et al. Nucleic acid detection with CRISPR-Cas13a/C2c2. Science (80-.). 356, 438-442 (2017).
10. Barnes, K. G. et al. Deployable CRISPR-Cas13a diagnostic tools to detect and report Ebola and Lassa virus cases in real-time. Nat. Commun. 11, (2020).
11. Granados-Riveron, J. T. & Aquino-Jarquin, G. CRISPR-Cas13 Precision Transcriptome Engineering in Cancer. Cancer Res. 4107-4114 (2018). doi:10.1158/0008-5472.CAN-18-0785
12. Freije, C. A. et al. Programmable Inhibition and Detection of RNA Viruses Using Cas13. Mol. Cell 76, 826-837.el1 (2019).
13. Cui, J., Techakriengkrai, N., Nedumpun, T. & Suradhat, S. Abrogation of PRRSV infectivity by CRISPR-Cas13b-mediated viral RNA cleavage in mammalian cells. Sci. Rep. 10, 9617 (2020).
14. Kushawah, G. et al. CRISPR-Cas13d Induces Efficient mRNA Knockdown in Animal Embryos. Dev. Cell 54, 805-817.e7 (2020).
15. Hendel, A. et al. Chemically modified guide RNAs enhance CRISPR-Cas genome editing in human primary cells. Nat. Biotechnol. 33, 985-989 (2015).
16. McMahon, M. A., Prakash, T. P., Cleveland, D. W., Bennett, C. F. & Rahdar, M. Chemically Modified Cpfl-CRISPR RNAs Mediate Efficient Genome Editing in Mammalian Cells. Mol. Ther. 26, 1228-1240 (2018).
17. Wessels, H. H. et al. Massively parallel Cas13 screens reveal principles for guide RNA design. Nat. Biotechnol. 38, 722-727 (2020).
18. Lee, K. et al. Synthetically modified guide RNA and donor DNA are a versatile platform for CRISPR-Cas9 engineering. Elife 6, 1-17 (2017).
19. Wienert, B., Shin, J., Zelin, E., Pestal, K. & Corn, J. E. In vitro-transcribed guide RNAs trigger an innate immune response via the RIG-I pathway. PLoS Biol. 16, 1-18 (2018).
20. Ryan, D. E. et al. Improving CRISPR-Cas specificity with chemical modifications in single-guide RNAs. Nucleic Acids Res. 46, 792-803 (2018).
21. Basila, M., Kelley, M. L. & Smith, A. V. B. Minimal 2′-O-methyl phosphorothioate linkage modification pattern of synthetic guide RNAs for increased stability and efficient CRISPR-Cas9 gene editing avoiding cellular toxicity. PLoS One 12, 1-19 (2017).
22. Abbott, T. R. et al. Development of CRISPR as an Antiviral Strategy to Combat SARS-CoV-2 and Influenza. Cell 181, 865-876.e12 (2020).
23. Arizti-Sanz, J. et al. Integrated sample inactivation, amplification, and Cas13-based detection of SARS-CoV-2. bioRxiv Prepr. 2, (2020).
24. Lai, M. M. C. Coronavirus leader-RNA-primed transcription: An alternative mechanism to RNA splicing. BioEssays 5, 257-260 (1986).
25. Kim, D. et al. The Architecture of SARS-CoV-2 Transcriptome. Cell 181, 914-921.e10 (2020).
26. Fozouni, P. et al. Amplification-free detection of SARS-CoV-2 with CRISPR-Cas13a and mobile phone microscopy. Cell 1-11 (2020).
27. Patchsung, M. et al. Clinical validation of a Cas13-based assay for the detection of SARS-CoV-2 RNA. Nat. Biomed. Eng. 4, (2020).
28. Joung J. et al. Detection of SARS-CoV-2 with SHERLOCK One-Pot Testing. N. Engl. J. Med. 2018-2021 (2020).

What is embodimented is:

1. A modified crRNA comprising a spacer sequence and a direct repeat sequence, wherein the crRNA comprises one or more chemically modified nucleotides.

2. The modified crRNA of embodiment 1, wherein the spacer sequence comprises at least 23 nucleotides, and is complementary to a target RNA sequence.

3. The modified crRNA of embodiment 1, wherein the direct repeat sequence is 5′ to the spacer sequence.

4. The modified crRNA of any of embodiments 1 to 3, wherein the chemically modified nucleotides are located at the 5′ end of the crRNA or the 3′ end of the crRNA.

5. The modified crRNA of any of embodiments 1 to 4, comprising chemically modified nucleotides throughout the crRNA.

6. The modified crRNA of any one of embodiments 1 to 5, comprising 1, 2, 3, 4, or 5 modified nucleotides at the 3′ end of the spacer sequence.

7. The modified crRNA of any one of embodiments 1 to 5, comprising 1, 2, 3, 4, or 5 modified nucleotides located downstream of the 3′ end of the spacer sequence.

8. The modified crRNA of any of embodiments 1 to 7, wherein the chemically modified nucleotide comprises a modification is selected from 2′-O-methyl (M), 3′phosphorothioate (S), 2′O-methyl-3′-phosphorothioate (MS) and 3′ Inverted thymine (invT).

9. The modified crRNA of any of embodiments 1 to 8, wherein the chemical modification comprises a 2′O-methyl (M) modification.

10. The modified crRNA of any of embodiments 1 to 9, wherein the chemical modification comprises a 3′phosphorothioate (S) modification.

11. The modified crRNA of any of embodiments 1 to 10, wherein the chemical modification comprises a 2′O-methyl-3′-phosphorothioate (MS) modification.

12. The modified crRNA of any of embodiments 1 to 11, wherein the chemical modification comprises a 3′ inverted thymine (invT) modification.

13. A method of enhancing the stability of a crRNA, comprising modifying a crRNA comprising a spacer sequence and a direct repeat sequence, with one or more chemically modified nucleotides.

14. The method of embodiment 13, wherein the one or more chemically modified nucleotides comprises a chemical modification selected from 2′-O-methyl (M), 3′phosphorothioate (S), 2′O-methyl-3′-phosphorothioate (MS) and 3′ inverted thymine (invT).

15. The method of embodiment 13 or 14, wherein the spacer sequence comprises at least 23 nucleotides, and is complementary to a target RNA sequence.

16. The method of any one of embodiments 13 to 15, wherein the direct repeat sequence is 5′ to the spacer sequence.

17. The method of any one of embodiments 13 to 16, wherein the chemically modified nucleotides are located at the 5′ end of the crRNA or the 3′ end of the crRNA.

18. The method of any one of embodiments 13 to 17, comprising chemically modified nucleotides throughout the crRNA.

19. The method of any one of embodiments 13 to 17, comprising 1, 2, 3, 4, or 5 modified nucleotides at the 3′ end of the spacer sequence.

20. The method of any one of embodiments 13 to 17, comprising 1, 2, 3, 4, or 5 modified nucleotides located downstream of the 3′ end of the spacer sequence.

21. The method of any one of embodiments 13 to 20, wherein the chemically modified nucleotide comprises a modification is selected from 2′-O-methyl (M), 3′phosphorothioate (S), 2′O-methyl-3′-phosphorothioate (MS) and 3′ Inverted thymine (invT).

22. The method of any one of embodiments 13 to 21, wherein the chemical modification comprises a 2′O-methyl (M) modification.

23. The method of any one of embodiments 13 to 22, wherein the chemical modification comprises a 3′phosphorothioate (S) modification.

24. The method of any one of embodiments 13 to 23, wherein the chemical modification comprises a 2′O-methyl-3′-phosphorothioate (MS) modification.

25. The method of any one of embodiments 13 to 24, wherein the chemical modification comprises a 3′ Inverted thymine (invT) modification.

26. A method of enhancing modulation of gene transcripts in a cell, the method comprising: introducing into the cell
(a) the modified crRNA of any of embodiments 1 to 12; and
(b) a Cas13 polypeptide, an mRNA encoding a Cas13 polypeptide, and/or a recombinant expression vector comprising a nucleotide sequence encoding a Cas13 polypeptide,
wherein the modified crRNA guides the Cas13 polypeptide to the target RNA sequence, and wherein the modified crRNA induces regulation of the target RNA with an enhanced activity relative to a corresponding unmodified crRNA.

27. The method of embodiment 26, wherein the enhanced activity comprises increased stability of the modified crRNA and/or increased specificity of the modified crRNA for the target RNA.

28. The method of embodiment 26 or 27, wherein the target RNA is mRNA and the modulation comprisesdownregulation of protein expression of the target mRNA.

29. A composition comprising the modified crRNA of any one of embodiments 1 to 12, wherein the spacer sequence comprises a sequence complementary to the SARS-CoV-2 leader sequence.

30. A method of treating COVID-19 infection, the method comprising introducing into a subject having, or suspected of having, COVID-19 infection:
(a) the composition according to embodiment 29; and
(b) a Cas13 polypeptide, an mRNA encoding a Cas13 polypeptide, and/or a recombinant expression vector comprising a nucleotide sequence encoding a Cas13 polypeptide,
wherein the modified crRNA guides the Cas13 polypeptide to the SARS-CoV-2 leader sequence, and wherein the modified crRNA induces knockdown of the SARS-CoV-2 virus, thereby treating COVID-19 infection.

31. A method of modulating gene transcripts in a primary cell, the method comprising: introducing into the cell
(a) the modified crRNA of any of embodiments 1 to 12; and
(b) a Cas13 polypeptide, an mRNA encoding a Cas13 polypeptide, and/or a recombinant expression vector comprising a nucleotide sequence encoding a Cas13 polypeptide,
wherein the modified crRNA guides the Cas13 polypeptide to the target RNA sequence, and wherein the modified crRNA induces regulation of the target RNA with an enhanced activity relative to a corresponding unmodified crRNA.

32. The method of embodiment 31, wherein the primary cell is cultured in vitro for use in ex vivo therapy.

33. The method of embodiment 31, wherein the primary cell is contacted in vivo with the modified crRNA of (a) and the Cas13 polypeptide, an mRNA encoding a Cas13 polypeptide, and/or a recombinant expression vector comprising a nucleotide sequence encoding a Cas13 polypeptide of (b).

34. The method of any of embodiments 31 to 33, wherein the primary cell is a stem cell or an immune cell.

35. The method of embodiment 34, wherein the primary cell is a T-cell.

36. The method of embodiment 35, wherein the T-cell is a CD4+ or CD8+.
